(19) United States
(12) Patent Application Publication
(10) Pub. No.: US 2022/0142568 A1
(43) Pub. Date: May 12, 2022

(54) OPTICAL DEVICE AND ENDOSCOPE SYSTEM

(71) Applicant: OLYMPUS CORPORATION, Tokyo (JP)

(72) Inventors: Yasuo SASAKI, Tokyo (JP); Hiroyuki KAMEE, Tokyo (JP)

(73) Assignee: OLYMPUS CORPORATION, Tokyo (JP)

(21) Appl. No.: 17/584,875

(22) Filed: Jan. 26, 2022

Related U.S. Application Data
(63) Continuation of application No. PCT/JP2019/029982, filed on Jul. 31, 2019.

(51) Int. Cl. A61B 5/00; A61B 1/07; A61B 1/06; A61B 1/05; A61B 1/005

(52) U.S. Cl. CPC A61B 5/4887; A61B 1/07; A61B 1/0625; A61B 1/05; A61B 1/005; A61B 1/0638

(57) ABSTRACT

An optical device includes a light source unit and a body unit. The light source unit includes a first light source configured to emit first irradiation light, a second light source configured to emit second irradiation light, a light source control unit, and a light collecting unit. The body unit includes an insertion section. The insertion section includes a light guide member, an optical system, an optical filter, a first imager configured to output image information of a subject, and a second imager configured to output distance information from the optical system to the subject. In the second irradiation light, light intensity is temporally modulated, and the first irradiation light and the second irradiation light are emanated from the insertion section. First measurement light includes light in the same wavelength band as a part of the wavelength band of the first irradiation light, and second measurement light includes light in the same wavelength band as the wavelength band of the second irradiation light, and error information included in the distance information is reduced.

### CROSS REFERENCES

The present application is a continuation application of International Application No. PCT/JP2019/029982 filed on Jul. 31, 2019, the entire contents of which are incorporated herein by reference.

### BACKGROUND OF INVENTION

### Technical Field

The present disclosure relates to an optical device and an endoscope system.

### Description of the Related Art

In gastroendoscopy, an image of the interior of the stomach is acquired. In intestinal endoscopy, an image of the interior of the bowel is acquired. It is possible to find a lesion such as a tumor from the acquired image.

Once a lesion is found, a course of treatment for the lesion is determined. In determination of a course of treatment, the size of the lesion area matters. It is therefore important to grasp the size of the lesion area precisely.

In order to grasp the size of the lesion area precisely, it is necessary to measure the distance from the endoscope to the lesion area precisely. For example, it is possible to use parallax in measurement of the distance. In measurement using parallax, however, the longer the distance from the endoscope to the lesion area is, the smaller the parallax is. When the parallax is small, the measurement accuracy is reduced. Therefore, when the distance from the endoscope to the lesion area is long, it is difficult to measure the distance from the endoscope to the lesion area precisely.

As another measurement method, a measurement method called the Time of Flight method (hereinafter referred to as “TOF method”) is disclosed in Japanese Patent Application Laid-open No. 2014-138691. In the TOF method, light with light intensity temporally modulated and a TOF imager are used.

FIG. 26A, FIG. 26B, and FIG. 26C are diagrams illustrating the measurement principle of the TOF method. FIG. 26A is a diagram illustrating light intensity in a white light source, FIG. 26B is a diagram illustrating light intensity in a TOF light source, and FIG. 26C is a diagram illustrating a state of measurement.

In an endoscope, a white light source is used for illumination of a target. For example, a white LED, a white LD, a halogen lamp, or a xenon lamp is used as the white light source. In the white LED, a plurality of LEDs are used, or an LED and a phosphor are used. In the white LD, a plurality of LDs are used, or an LD and a phosphor are used.

As illustrated in FIG. 26A, illumination light L_{w} in a wavelength band ΔλL_{w} is emitted from the white light source. The wavelength band ΔλL_{w} includes wavelengths in the visible range. By using an optical filter, it is possible to extract light in a wavelength band narrower than the wavelength band ΔλL_{w} from the white light source. It is possible to use the light in a narrow wavelength band, for example, for narrow band imaging (NBI).

Furthermore, as illustrated in FIG. 26A, in the illumination light L_{w}, light intensity IL_{w} does not change over time. That is, light with light intensity not temporally modulated is used as the illumination light L_{w}. However, light with light intensity temporally modulated (hereinafter referred to as “continuous pulsed light”) may be used as the illumination light L_{w}.

In the continuous pulsed light, the light intensity periodically changes over time. It is possible to use rectangular pulsed light or sinusoidal pulsed light as the continuous pulsed light. The rectangular pulsed light is continuous pulsed light in which change in light intensity is represented by rectangular waves. The sinusoidal pulsed light is continuous pulsed light in which change in light intensity is represented by sinusoidal waves.

In modulation of light intensity, turning-on and turning-off of light are repeated. In the white light source, for example, the period of repetition is 1 μs or longer. The frequency of repetition is 1 MHz or lower. Pulse width modulation is often used in modulation of light intensity. In the pulse width modulation, it is possible to change the light intensity by changing the pulse width at a time of turning-on.

In the TOF light source, as illustrated in FIG. 26B, illumination light L_{TOF} in a wavelength band ΔλL_{TOF} is emitted. The wavelength band ΔλL_{TOF} is usually in the near infrared region. The bandwidth of the wavelength band ΔλL_{TOF} is narrower than the bandwidth of the wavelength band ΔλL_{w}.

The reasons why the near infrared region is selected include invisibility to human eyes (measurement is unaware), relative inexpensiveness of light sources, and availability of common silicon imagers.

Furthermore, in the illumination light L_{TOF}, as illustrated in FIG. 26B, the light intensity IL_{TOF} changes over time. For example, in the illumination light L_{TOF}, the light intensity is temporally modulated at a frequency of 10 MHz to 100 MHz.

In measurement by the TOF method, continuous pulsed light is used as illumination light. It is possible to use rectangular pulsed light or sinusoidal pulsed light as the continuous pulsed light.

When continuous pulsed light is rectangular pulsed light, a distribution shape of light intensity of one pulsed light (hereinafter referred to as “pulse shape”) is rectangular. In the following, the measurement principle will be described on the premise that the pulse shape is rectangular.

In the measurement by the TOF method, light from a light source to a target is compared with light from the target to a photodetector. An optical element, for example, a lens is usually disposed between the light source and the target. In addition, an optical element is also disposed between the target and the photodetector.

When an optical element is disposed in an optical path, light passing through the optical element is influenced by the optical element. However, it is possible to explain the measurement principle without explaining the influence of the optical element. Thus, the measurement principle will be explained based on a state in which an optical element is not disposed.

As illustrated in FIG. 26C, the target is illuminated with illumination light L_{ILL}. Return light L_{R} is emanated from the target. The return light L_{R} is light reflected by the target or light scattered by the target. The return light L_{R} is detected by a TOF imager (not illustrated).

Since the illumination light L_{ILL} is pulsed light, the return light L_{R} is also pulsed light. Pulsed light emitted from a light source is reflected by the target and detected by the TOF imager. Thus, when attention is paid to one pulsed light, there is a difference between the time when pulsed light is emitted from the light source and the time when the pulsed light reaches the TOF imager.

FIG. 27A and FIG. 27B are diagrams illustrating the measurement principle of the TOF method. FIG. 27A is a diagram illustrating a case where the distance to a target is short, and FIG. 27B is a diagram illustrating a case where the distance to a target is long.

In the TOF imager, two or more gate signals are used. In FIG. 27 A and FIG. 27B, a first signal GATE1 and a second signal GATE2 are used as the gate signals. In the TOF imager, charge is accumulated in a first accumulator when the first signal GATE1 is High. Furthermore, charge is accumulated in a second accumulator when the second signal GATE2 is High, in the same manner as the first signal GATE1.

The illumination light L_{ILL} and the return light L_{R} are both pulsed light. The pulse shape in the illumination light L_{ILL} and the pulse shape in the return light L_{R} are both rectangular. Then, the illumination light L_{ILL} and the return light L_{R} are compared at a rising portion of the pulsed shape.

When the distance to a target is short, as illustrated in FIG. 27A, there is a difference Δtn between the illumination light L_{ILL} and the return light L_{R}. In this case, charge is accumulated in the first accumulator during time t1n while the first signal GATE1 is High. A signal I1n is obtained from the accumulated charge. Charge is accumulated in the second accumulator during time t2n while the second signal GATE2 is High. A signal I2n is obtained from the accumulated charge.

When the distance to a target is long, as illustrated in FIG. 27B, there is a difference Δtf between the illumination light L_{ILL} and the return light L_{R}. In this case, charge is accumulated in the first accumulator during time t1f while the first signal GATE1 is High. A signal I1f is obtained from the accumulated charge. Charge is accumulated in the second accumulator during time t2f while the second signal GATE2 is High. A signal I2f is obtained from the accumulated charge.

The relation between time and signal is as follows.

When the distance to a target is short: t2n<t1n, I2n<I1n

When the distance to a target is long: t1f<t2f, I1f<I2f

As just described, when the distance to a target changes, the ratio between a signal obtained when the first signal GATE1 is High and a signal obtained when the second signal GATE2 is High changes. Thus, it is possible to measure the distance to the target from two signals.

The TOF imager includes a plurality of light-receiving portions. It is possible to measure the distance to a target with each of the light-receiving portions. Thus, it is possible to grasp the size of the target.

In the TOF method, rectangular pulsed light or sinusoidal pulsed light is used for measurement of the distance. If the pulse shape in the return light L_{R} differs from the pulse shape in the illumination light L_{ILL}, accurate measurement is difficult whether rectangular pulsed light is used or sinusoidal pulsed light is used.

As illustrated in FIG. 26C, the illumination light L_{ILL} emitted from the light source reaches the TOF imager in the form of the return light L_{R}. However, an optical element is typically disposed between the light source and the target and between the target and the photodetector as described above.

Hence, whether rectangular pulsed light is used or sinusoidal pulsed light is used, the pulse shape changes under the influence of the optical element. Furthermore, the pulse shape also changes under the influence of the target. The change in pulse shape means that error information is added to the distance information.

Sinusoidal pulsed light incident on the optical system is emanated from the optical system. At this moment, sinusoidal pulsed light lagging behind in phase from the incident sinusoidal pulsed light is emanated from the optical system in some cases. The phase lag occurs because pulsed light having a time delay in the optical system is overlapped. Consequently, error information may be added to the distance information due to the phase lag. Thus, this phase lag is also included in the change in pulse shape.

### SUMMARY

An optical device according to at least some embodiments of the present disclosure includes:

a light source unit; and a body unit, wherein

the light source unit includes

a first light source configured to emit first irradiation light,

a second light source configured to emit second irradiation light,

a light source control unit configured to control the first light source and the second light source, and

a light collecting unit on which the first irradiation light and the second irradiation light are incident,

the body unit includes a rigid and tubular insertion section or a flexible and tubular insertion section,

the insertion section includes

a light guide member formed of a transparent medium having a refractive index larger than 1,

an optical system on which return light from a subject is incident,

a first imager configured to output image information of the subject based on first measurement light, and

a second imager configured to output distance information from the optical system to the subject based on second measurement light,

in the second irradiation light, light intensity is temporally modulated,

the light guide member has an incident end face positioned on a light collecting unit side and an exit end face positioned on a subject side,

third irradiation light emanated from the light collecting unit is emanated from the insertion section toward the subject,

the first measurement light includes light in the same wavelength band as a part of a wavelength band of the first irradiation light,

the second measurement light includes light in the same wavelength band as a wavelength band of the second irradiation light, and

error information included in the distance information is reduced.

An endoscope system according to at least some embodiments of the present disclosure includes:

the optical device described above; and a processing device, wherein

the processing device includes an assistance information generator configured to generate assistance information,

the assistance information is generated based on the image information and the distance information, and

the assistance information includes information on position and information on shape of a lesion candidate region, and a length between necessary points calculated from the distance information based on the information.

An endoscope system according to at least some embodiments of the present disclosure includes:

the optical device described above; and a processing device, wherein

an image for observation of the subject is generated based on the image information,

a distance or a distance and an inclination in a pixel of the image for observation are complemented and estimated based on the distance information, and

length information is acquired from a result of the estimation.

### BRIEF DESCRIPTION OF THE DRAWINGS

FIG. 1A and FIG. 1B are diagrams illustrating an optical device of the present embodiment;

FIG. 2 is a diagram illustrating an optical device of the present embodiment;

FIG. 3A and FIG. 3B are diagrams illustrating a light source unit;

FIG. 4 is a diagram illustrating a wavelength band of irradiation light;

FIG. 5A, FIG. 5B, FIG. 5C, and FIG. 5D are diagrams illustrating the light source unit and wavelengths of irradiation light;

FIG. 6 is a diagram illustrating the light source unit;

FIG. 7 is a diagram illustrating the light source unit;

FIG. 8A and FIG. 8B are diagrams illustrating the wavelength band of first irradiation light and the wavelength band of second irradiation light;

FIG. 9A and FIG. 9B are diagrams illustrating irradiation light and measurement light;

FIG. 10A and FIG. 10B are diagrams illustrating irradiation light and measurement light;

FIG. 11A, FIG. 11B, and FIG. 11C are diagrams illustrating the wavelength band of first irradiation light and the wavelength band of second irradiation light;

FIG. 12A and FIG. 12B are diagrams illustrating irradiation light and measurement light;

FIG. 13A and FIG. 13B are diagrams illustrating irradiation light and measurement light;

FIG. 14 is a diagram illustrating measurement light;

FIG. 15A and FIG. 15B are diagrams illustrating measurement light;

FIG. 16A, FIG. 16B, and FIG. 16C are diagrams illustrating an exit region of an incident end face;

FIG. 17 is a diagram illustrating an optical device of the present embodiment;

FIG. 18 is a diagram illustrating an optical device of the present embodiment;

FIG. 19A, FIG. 19B, and FIG. 19C are diagrams illustrating a first example of an exit end face and an exit region;

FIG. 20 is a diagram illustrating a second example of the exit end face;

FIG. 21A, FIG. 21B, FIG. 21C, and FIG. 21D are diagrams illustrating a light guide member;

FIG. 22A, FIG. 22B, FIG. 22C, and FIG. 22D are diagrams illustrating a light guide member;

FIG. 23A and FIG. 23B are diagrams illustrating an optical device of the present embodiment and an incident region;

FIG. 24 is a diagram illustrating an optical device of the present embodiment;

FIG. 25 is a diagram illustrating an endoscope system of the present embodiment;

FIG. 26A, FIG. 26B, and FIG. 26C are diagrams illustrating the measurement principle of the TOF method;

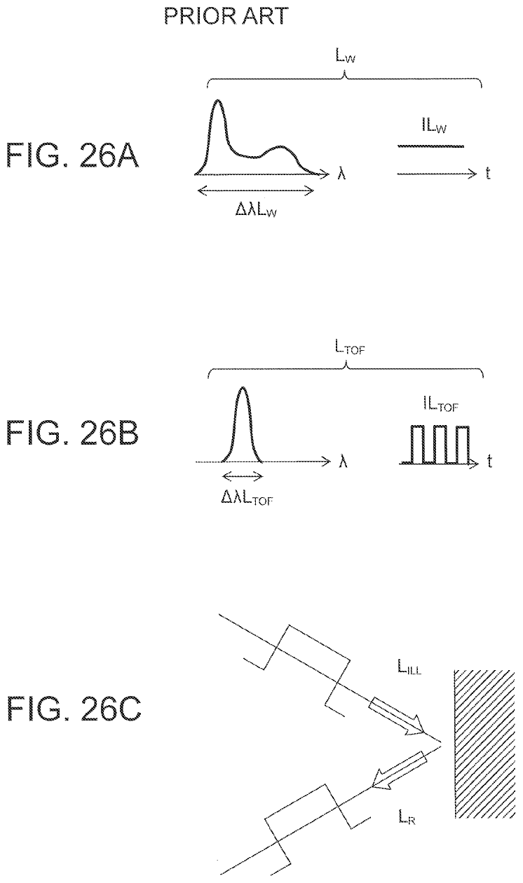

and

FIG. 27A and FIG. 27B are diagrams illustrating the measurement principle of the TOF method.

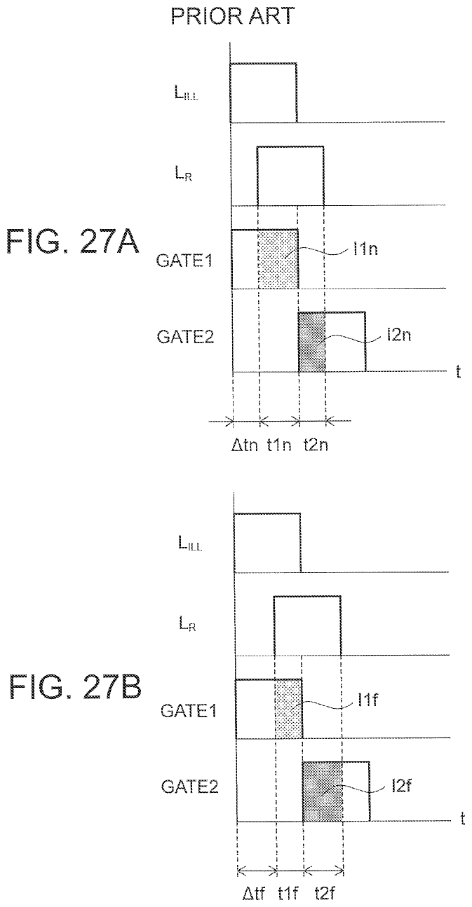

### DETAILED DESCRIPTION

Prior to the explanation of examples, action and effect of embodiments according to certain aspects of the present disclosure will be described below. In the explanation of the action and effect of the embodiments concretely, the explanation will be made by citing concrete examples. However, similar to a case of the examples to be described later, aspects exemplified thereof are only some of the aspects included in the present disclosure, and there exists a large number of variations in these aspects. Consequently, the present disclosure is not restricted to the aspects that will be exemplified.

### (Optical Device 1 of the Present Embodiment)

An optical device of the present embodiment includes a light source unit and a body unit. The light source unit includes a first light source configured to emit first irradiation light, a second light source configured to emit second irradiation light, a light source control unit configured to control the first light source and the second light source, and a light collecting unit on which the first irradiation light and the second irradiation light are incident. The body unit includes a rigid and tubular insertion section or a flexible and tubular insertion section. The insertion section includes a light guide member formed of a transparent medium having a refractive index larger than 1, an optical system on which return light from a subject is incident, a first imager configured to output image information of the subject based on first measurement light, and a second imager configured to output distance information from the optical system to the subject based on second measurement light. In the second irradiation light, light intensity is temporally modulated. The light guide member has an incident end face positioned on the light collecting unit side and an exit end face positioned on the subject side. Third irradiation light emanated from the light collecting unit is emanated from the insertion section toward the subject. The first measurement light includes light in the same wavelength band as a part of a wavelength band of the first irradiation light, the second measurement light includes light in the same wavelength band as a wavelength band of the second irradiation light, and error information included in the distance information is reduced.

### (Optical Device 1: First Example)

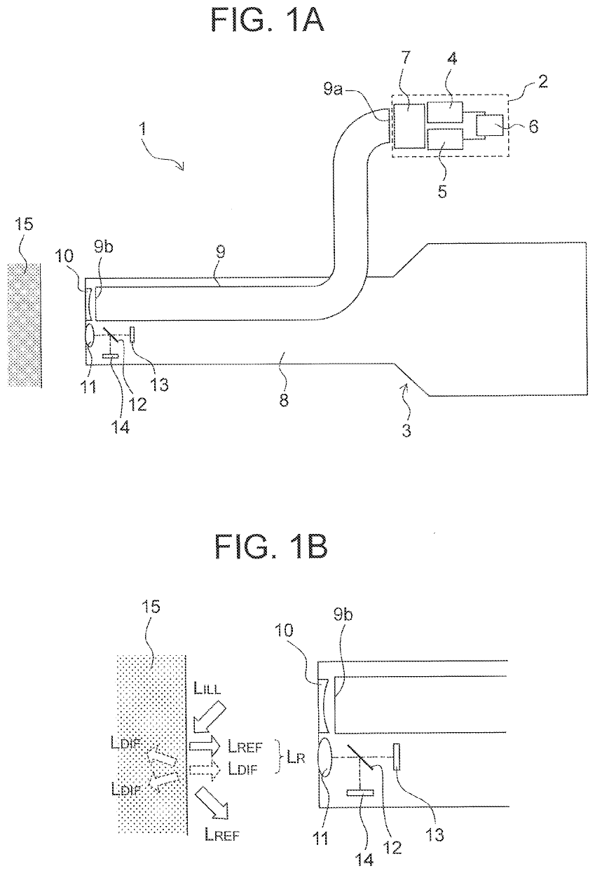

FIG. 1A and FIG. 1B are diagrams illustrating an optical device. FIG. 1A is a diagram illustrating the entire optical device. FIG. 1B is a diagram illustrating an end of the optical device.

As illustrated in FIG. 1A, an optical device 1 includes a light source unit 2 and a body unit 3. In the optical device 1, the light source unit 2 is disposed at a place away from the body unit 3.

The light source unit 2 includes a first light source 4, a second light source 5, a light source control unit 6, and a light collecting unit 7. First irradiation light is emitted from the first light source 4. Second irradiation light is emitted from the second light source 5.

The light source control unit 6 controls the first light source 4 and the second light source 5. In the light source control unit 6, for example, turning-on and turning-off of the first light source 4, turning-on and turning-off of the second light source 5, adjustment of light intensity of the first irradiation light, and adjustment of light intensity of the second irradiation light are performed.

The first irradiation light and the second irradiation light are incident on the light collecting unit 7. A specific configuration of the light collecting unit 7 will be described later. Third irradiation light is emanated from the light collecting unit 7. In the third irradiation light, light having the same wavelength band as a part of the wavelength band of the first irradiation light and the second irradiation light are included, or the first irradiation light and the second irradiation light are included.

The body unit 3 includes an insertion section 8. The insertion section 8 is formed of a rigid and tubular member or a flexible and tubular member. The insertion section 8 includes a light guide member 9, an optical system 11, an optical filter 12, a first imager 13, and a second imager 14. The insertion section 8 may further include a lens 10.

The insertion section 8 includes a coaxial optical system. In the coaxial optical system, one optical path is formed from the optical system 11 to the optical filter 12. Two optical paths are formed by the optical filter 12. The first imager 13 is disposed on one of the optical paths, and the second imager 14 is disposed on the other optical path.

For example, it is possible to use a dichroic mirror or a half mirror as the optical filter 12. For example, it is possible to use a CCD or a CMOS as the first imager 13. A TOF imager is used as the second imager 14.

The light guide member 9 is formed of a transparent medium having a refractive index larger than 1. It is possible to use a single fiber or a fiber bundle as the light guide member 9. It is possible to use a relay optical system instead of the light guide member 9.

As described later, the third irradiation light is incident on the light guide member 9. The third irradiation light propagates through the interior of the light guide member 9 and is emanated from the light guide member 9. As a result, the third irradiation light is emanated from the insertion section 8. A subject 15 is irradiated with the third irradiation light. The subject 15 is thus illuminated.

Return light from the subject is incident on the optical system 11. The return light includes reflected light directed toward the optical system 11 and scattered light directed toward the optical system 11. The return light will be described later.

The return light incident on the optical system 11 reaches the optical filter 12. The return light is split into transmitted light and reflected light at the optical filter 12. The transmitted light is the first measurement light, and the reflected light is the second measurement light.

The first measurement light is incident on the first imager 13. Image information of the subject is output from the first imager 13, based on the first measurement light. The second measurement light is incident on the second imager 14. Distance information from the optical system to the subject is output from the second imager 14, based on the second measurement light.

The first measurement light and the second measurement light are included in light when the third irradiation light returns from the subject. Each of the wavelength band of the first measurement light and the wavelength band of the second measurement light includes a part of the wavelength band of the third irradiation light.

The first light source 4 is a light source for image acquisition. In the image acquisition, it is preferable that brightness information of the subject 15 be acquired. For example, brightness information is obtained by acquiring an image with white light illumination or acquiring an image by NBI. A case where an image is acquired with white light illumination will be described below.

It is possible to make the first irradiation light white light. For example, it is possible to use a white LED, a white LD, a halogen lamp, or a xenon lamp as the first light source 4. White light includes light having continuous spectra and light having discontinuous spectra.

The light having discontinuous spectra includes a plurality of wavelengths at which light intensity is substantially zero. The wavelength band of the light having discontinuous spectra is determined by the shortest wavelength and the longest wavelength among the wavelengths at which light intensity is substantially zero.

The second light source 5 is a light source for TOF. Thus, the second irradiation light is monochrome light or semi-monochrome light (hereinafter referred to as “narrow-band light”). For example, an LD or an LED is used as the second light source 5.

Since the first irradiation light is white light, the wavelength band of the first irradiation light is wider than the wavelength band of the second irradiation light. Furthermore, light with light intensity not temporally modulated or continuous pulsed light is used as the first irradiation light. On the other hand, continuous pulsed light is used as the second irradiation light.

The light guide member 9 has an incident end face 9a positioned on the light collecting unit 7 side and an exit end face 9b positioned on the subject 15 side.

The incident end face 9a faces the light collecting unit 7. As described above, the third irradiation light is emanated from the light collecting unit 7. Thus, the third irradiation light is incident on the incident end face 9a. The third irradiation light incident on the light guide member 9 propagates through the interior of the light guide member 9 and reaches the exit end face 9b.

The third irradiation light is emanated from the exit end face 9b. The lens 10 is disposed on the exit end face 9b side. The lens 10 faces the subject 15. Thus, the subject 15 is irradiated with the third irradiation light through the lens 10. As a result, the subject 15 is illuminated with the third irradiation light.

The return light will be described. When the subject 15 is irradiated with illumination light, light reflected in the vicinity of a surface of the subject 15 and light reaching the interior of the subject 15 occur. The light reaching the interior of the subject 15 is scattered in the interior of the subject. Part of the scattered light is emanated from the subject 15 and incident on the optical system 11 together with the reflected light. The return light thus includes reflected light and scattered light.

As illustrated in FIG. 1B, the subject 15 is illuminated with illumination light L_{ILL}. The illumination light L_{ILL} includes the first irradiation light and the second irradiation light.

When the subject 15 is body tissue, reflected light L_{REF} and scattered light L_{DIF} occur in the subject 15. The reflected light L_{REF} is light produced when the illumination light L_{ILL} is reflected by the subject 15. The scattered light L_{DIF} is light produced when the illumination light L_{ILL} is scattered by the subject 15.

The lens 10 and the optical system 11 are disposed side by side. In this case, the subject 15 is obliquely irradiated with the illumination light L_{ILL}. That is, the illumination light L_{ILL} travels from the outside toward the inside of the field of view of the optical system 11.

The illumination light L_{ILL} includes light rays at various angles. In the reflected light L_{REF}, most of the reflected light is directed toward the outside of the field of view of the optical system 11, and the remaining reflected light is directed toward the optical system 11. On the other hand, the scattered light is directed in every direction. In the scattered light L_{DIF}, part of the scattered light is directed toward the optical system 11.

The return light L_{R} from the subject 15 is incident on the optical system 11. The return light L_{R} includes the reflected light L_{REF} directed toward the optical system 11 and the scattered light L_{DIF} directed toward the optical system 11. The return light L_{R} is split into transmitted light and reflected light by the optical filter 12. The transmitted light is the first measurement light, and the reflected light is the second measurement light.

The transmitted light and the reflected light both include the reflected light L_{REF} and the scattered light L_{DIF}. Thus, the first measurement light and the second measurement light both include the reflected light L_{REF} and the scattered light L_{DIF}.

The first measurement light includes light in an overlapping wavelength band. The overlapping wavelength band is the same wavelength band as the wavelength band of the first irradiation light.

When the overlapping wavelength band matches with a part of the wavelength band of the first irradiation light, the wavelength band of the first measurement light is different from the wavelength band of the first irradiation light. When the overlapping wavelength band matches with the entire wavelength band of the first irradiation light, the wavelength band of the first measurement light is the same as the wavelength band of the first irradiation light.

When the overlapping wavelength band matches with a part of the wavelength band of the first irradiation light, the wavelength band of the first measurement light is the same as the wavelength band in which a particular wavelength band is missing from the wavelength band of the first irradiation light. If the missing particular wavelength band is narrow, it is possible to consider the wavelength band of the first measurement light to be the same as the wavelength band of the first irradiation light.

As described above, the first irradiation light is white light. When the wavelength band of the first measurement light is the same as the wavelength band of the first irradiation light, the first measurement light is white light. When the wavelength band of the first measurement light is different from the wavelength band of the first irradiation light, it is possible to consider the first measurement light to be white light by narrowing the missing particular wavelength band.

In the first imager 13, an optical image of the subject illuminated with white light is formed. Thus, image information at a time of illuminating with white light is output from the first imager 13.

The wavelength band of the second measurement light includes light in the same wavelength band as the wavelength band of the second irradiation light. Thus, the wavelength band of the second measurement light is different from the wavelength band of the second irradiation light or the same as the wavelength band of the second irradiation light.

As described above, the second irradiation light is narrow-band light. When the wavelength band of the second measurement light is the same as the wavelength band of the second irradiation light, the second measurement light is narrow-band light. When the wavelength band of the second measurement light is different from the wavelength band of the second irradiation light, it is possible to make the second measurement light into narrow-band light by eliminating light other than the second irradiation light.

In the second imager 14, an optical image of the subject illuminated with narrow-band light is formed. In addition, the second measurement light includes light with light intensity temporally modulated. Thus, distance information from the optical system 11 to the subject is output from the second imager 14.

### (Optical Device 1: Second Example)

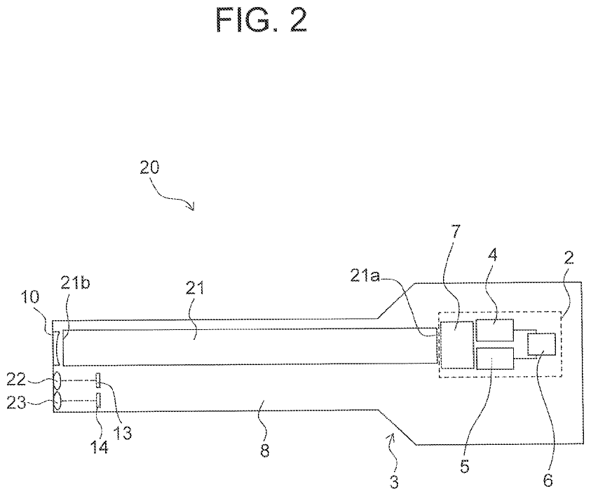

FIG. 2 is a diagram illustrating an optical device. The same component as that in FIG. 1A is denoted by the same numeral and a description thereof is omitted.

An optical device 20 includes the light source unit 2 and the body unit 3. In the optical device 20, the light source unit 2 is disposed in the interior of the body unit 3. The insertion section 8 includes a light guide member 21. The light guide member 21 has an incident end face 21a positioned on the light collecting unit 7 side and an exit end face 21b positioned on the subject side.

The insertion section 8 includes a parallel optical system. The parallel optical system includes a first optical system 22 and a second optical system 23. The first optical system 22 and the second optical system 23 are disposed side by side. Two optical paths are formed by the first optical system 22 and the second optical system 23. The first imager 13 is disposed on the optical path of the first optical system 22, and the second imager 14 is disposed on the optical path of the second optical system 23.

In the parallel optical system, two optical systems are disposed side by side. Hence, the outer diameter of a lens in one optical system is small, compared with the coaxial optical system. As a result, the resolving power of the optical system is degraded in the parallel optical system compared with the coaxial optical system. Furthermore, in the parallel optical system, the size of a pencil of light incident on one optical system is small, compared with the coaxial optical system.

FIG. 1A, FIG. 1B, and FIG. 2 are schematic diagrams of the optical device. Thus, in FIG. 1A, FIG. 1B, and FIG. 2, one light guide member, one incident end face, and one exit end face are depicted.

However, the number of light guide members is not limited to one. The optical device may include a plurality of light guide members. Furthermore, the number of incident end faces is not limited to one. The optical device may have a plurality of incident end faces. The number of exit end faces is not limited to one. The optical device may have a plurality of exit end faces.

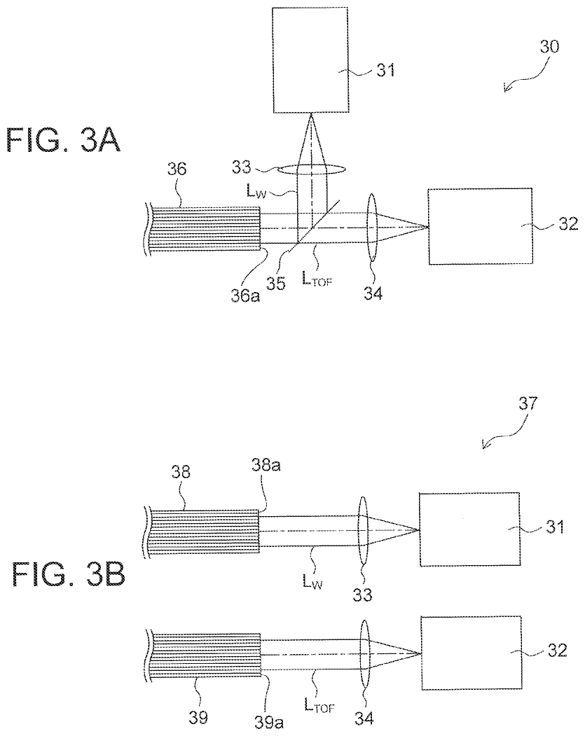

The light source unit will be described. FIG. 3A and FIG. 3B are diagrams illustrating the light source unit. FIG. 3A is a diagram illustrating a first example of the light source unit. FIG. 3B is a diagram illustrating a second example of the light source unit.

### (Light Source Unit: First Example)

A light source unit 30 is a coaxial incident-type light source unit. As illustrated in FIG. 3A, the light source unit 30 includes a first light source 31, a second light source 32, a lens 33, a lens 34, a dichroic mirror 35, and a light guide member 36. The light guide member 36 has an incident end face 36a. One light guide member is used in the light source unit 30.

In the light source unit 30, two illumination optical paths are formed. The first light source 31 and the lens 33 are disposed on one of the two illumination optical paths, and the second light source 32 and the lens 34 are disposed on the other illumination optical path. The dichroic mirror 35 is disposed at a position where the two illumination optical paths intersect.

First irradiation light L_{W} is emitted from the first light source 31. The first irradiation light L_{W} is white light. The first irradiation light L_{W} passes through the lens 33 and is incident on the dichroic mirror 35. Second irradiation light L_{TOF} is emitted from the second light source 32. The second irradiation light L_{TOF} is narrow-band light. The second irradiation light L_{TOF} passes through the lens 34 and is incident on the dichroic mirror 35.

The first irradiation light L_{W} is reflected by the dichroic mirror 35. The second irradiation light L_{TOF} is transmitted through the dichroic mirror 35. As a result, the third irradiation light travels through the same illumination optical path and is incident on the light guide member 36 from the incident end face 36a.

### (Light Source Unit: Second Example)

A light source unit 37 is a parallel incident-type light source unit. As illustrated in FIG. 3B, the light source unit 37 includes the first light source 31, the second light source 32, the lens 33, the lens 34, a light guide member 38, and a light guide member 39.

In the light source unit 37, two light guide members are used. The light guide member 38 has an incident end face 38a. The light guide member 39 has an incident end face 39a.

First irradiation light L_{W} is emitted from the first light source 31. The first irradiation light L_{W} is white light. The first irradiation light L_{W} passes through the lens 33 and is incident on the light guide member 38 from the incident end face 38a.

Second irradiation light L_{TOF} is emitted from the second light source 32. The second irradiation light L_{TOF} is narrow-band light. The second irradiation light L_{TOF} passes through the lens 34 and is incident on the light guide member 39 from the incident end face 39a.

The light source unit has been described above using point light sources as the first light source 31 and the second light source 32. However, surface light sources may be used as the first light source 31 and the second light source 32.

In this case, in the light source unit 30, a lens may be disposed between the dichroic mirror 35 and the incident end face 36a. In the light source unit 37, a lens may be disposed between the lens 33 and the incident end face 38a and between the lens 34 and the incident end face 39a. In this way, it is possible to form an image of the surface light source on the incident end face.

As described above, it is possible to use a coaxial optical system or a parallel optical system as the optical system. It is possible to use a coaxial incident-type light source unit or a parallel incident-type light source unit as the light source unit. Since there are two types of each of the light source unit and the optical system, four combinations of the light source unit and the optical system are made.

In the coaxial incident-type light source unit, the third irradiation light is incident on one fiber. In the parallel incident-type light source unit, the third irradiation light is split into the first irradiation light L_{W} and the second irradiation light L_{TOF}, which are incident on different fibers.

In the coaxial optical system, the return light L_{R} is incident on one optical system. In the parallel optical system, the return light L_{R} is incident on different optical systems.

In the optical device 1 and the optical device 20, error information included in the distance information is reduced. Hence, it is possible to accurately measure the distance to the target.

### (Optical Device 2 of the Present Embodiment)

In the optical device of the present embodiment, it is preferable that the second light source be used for reduction of error information, and the second irradiation light be light in a wavelength band on the shorter wavelength side than the infrared wavelength band commonly used.

When a subject is irradiated with the second irradiation light, return light L_{R}, that is, reflected light L_{REF} and scattered light L_{DIF} occur. The scattered light L_{DIF} is light scattered in the interior of the subject.

In the interior of the subject, scattered light occurs at every position that light reaches. The intensity of light reaching a place near a surface of the subject, that is, the intensity of light reaching a surface layer is high. Therefore, the scattered light occurring in the surface layer (hereinafter referred to as “surface layer scattered light”) has high light intensity. On the other hand, the intensity of light reaching a place away from a surface of the subject, that is, the intensity of light reaching a deep layer is low. Therefore, the scattered light occurring in the deep layer (hereinafter referred to as “deep layer scattered light”) has low light intensity.

Both the surface layer scattered light and the deep layer scattered light are light returning from the subject and therefore have distance information. The surface layer scattered light is scattered light occurring at a place near a surface of the subject. The surface layer scattered light has precise distance information and therefore can be used for acquisition of distance information.

On the other hand, the deep layer scattered light is not scattered light occurring at a place near a surface of the subject. It cannot be said that the deep layer scattered light has precise distance information and therefore is unable to be used for acquisition of distance information. That is, the deep layer scattered light should be considered as light causing error information. As just described, the first measurement light and the second measurement light include light having distance information and light causing error information.

The second measurement light is used for acquisition of distance information. Hence, if the second measurement light includes much light causing error information, the pulse shape becomes non-rectangular. If the pulse shape becomes non-rectangular, accurate measurement becomes difficult. In order to accurately measure the distance, light causing error information should be reduced.

The shorter the wavelength is, the more likely light is scattered. Hence, the shorter the wavelength of light is, the higher the proportion of the surface layer scattered light is. When the proportion of the surface layer scattered light is high, the amount of light reaching a place away from a surface of the subject is reduced. As a result, the amount of deep layer scattered light is reduced.

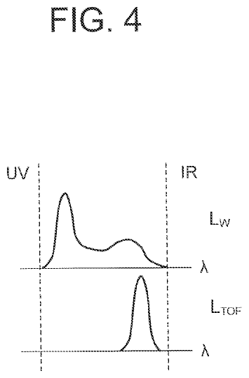

FIG. 4 is a diagram illustrating a wavelength band of irradiation light. As illustrated in FIG. 4, the wavelength band of the first irradiation light L_{W} is positioned between an ultraviolet wavelength band UV and an infrared wavelength band IR. The wavelength band of the second irradiation light L_{TOF} is narrower than the wavelength band of the first irradiation light L_{W}. Further, the wavelength band of the second irradiation light L_{TOF} is positioned on the shorter wavelength side than the infrared wavelength band IR.

As just described, in the optical device, light in a wavelength band on the shorter wavelength side than the infrared wavelength band (hereinafter referred to as “short-wavelength light”) is used for the second irradiation light L_{TOF}. It is therefore possible to diminish the deep layer scattered light, that is, light causing error information. As a result, it is possible to reduce error information.

When white light is used as the first irradiation light L_{W}, the band between the ultraviolet wavelength band UV and the infrared wavelength band IR indicates the wavelength band of white light. White light is light that looks white by the naked eye. It is possible to replace white light by visible light. The wavelength band of visible light is 400 nm to 700 nm.

### (Optical Device 2: Third Example)

In the optical device of the present embodiment, it is preferable that the second irradiation light include a wavelength band of 460 nm or more and 510 nm or less.

Oxyhemoglobin is contained in blood flowing through arteries. In veins, the ratio of deoxyhemoglobin in which oxygen is detached from oxyhemoglobin increases. The blood flows through arteries, capillaries, and veins in order. Capillaries are positioned in the middle between arteries and veins. Thus, capillaries include oxyhemoglobin and deoxyhemoglobin.

In a wavelength band of 460 nm or more and 510 nm or less, the absorption of light in oxyhemoglobin is small. When the absorption in oxyhemoglobin is small, return light from a region including arteries and capillaries increases as the loss of the second irradiation light due to absorption in oxyhemoglobin is small.

Furthermore, in this wavelength band, the absorption of light in deoxyhemoglobin is small. When the absorption of light in deoxyhemoglobin is small, return light from a region including veins and capillaries increases as the loss of the second irradiation light due to absorption in deoxyhemoglobin is small.

In this optical device, the second irradiation light includes light in a wavelength band of 460 nm or more and 510 nm or less. This wavelength band is a wavelength band shorter than near infrared light. Hence, when light in this wavelength band is used as irradiation light, it is possible to reduce error information because the scattered light from the interior of the subject can be reduced while the scattered light from the neighborhood of the surface can be relatively increased.

In addition, by using light in this wavelength band, it is possible to increase the return light in a region including a blood vessel. In the third example, therefore, it is possible to improve the accuracy in distance measurement in a region including a blood vessel.

When the subject has mucosa, there is a region in which capillaries are positioned in the neighborhood of the surface. Even in such a subject, measurement of the distance by scattered light from the neighborhood of a surface should be performed, but capillaries are distributed in the neighborhood of the surface depending on the location.

In such a subject, if the wavelength band of the second irradiation light includes a wavelength band in which absorption in oxyhemoglobin is large, the light intensity of return light is decreased in the region in which capillaries are distributed. Hence, the accuracy in distance measurement is deteriorated. If a wavelength band in which absorption in oxyhemoglobin is small is selected as the wavelength band of the second irradiation light, the light intensity of return light from the region in which blood vessels are distributed is increased. Thus, improvement in accuracy in distance measurement is expected.

When the wavelength band of the second irradiation light includes a wavelength band in which absorption in deoxyhemoglobin is large, the light intensity of return light from blood vessels is decreased. Hence, the accuracy in distance measurement is deteriorated. If a region in which absorption in oxyhemoglobin is small is selected as the wavelength band of the second irradiation light, the light intensity of return light from blood vessels is increased. Thus, improvement in accuracy in distance measurement is expected.

In this optical device, light in a wavelength band in which absorption in oxyhemoglobin is small and light in a wavelength band in which absorption in deoxyhemoglobin is small are included in the second irradiation light. Thus, by using the second irradiation light, it is possible to measure the distance to the subject more accurately.

### (Optical Device 2: Fourth Example)

In the optical device of the present embodiment, it is preferable that the second irradiation light be 460 nm or more and 510 nm or less.

In this optical device, light in a wavelength band of 460 nm or more and 510 nm or less is used as the second irradiation light. As described above, in this wavelength band, the absorption in oxyhemoglobin and the absorption in deoxyhemoglobin are small. Thus, in this optical device, light with small absorption in oxyhemoglobin and light with small absorption in deoxyhemoglobin are used for the second irradiation light. As a result, it is possible to measure the distance to the subject more accurately.

### (Optical Device 2: Fifth Example)

In the optical device of the present embodiment, it is preferable that the wavelength band of the second irradiation light include a wavelength band in which absorption in hemoglobin is large.

In the optical device of the present example, unlike the optical device of the third example and the optical device of the fourth example, light in a wavelength band in which absorption in hemoglobin is large is used as the second irradiation light.

The optical device of the present example is disadvantageous in that the SN ratio is slightly reduced because return light is extremely small. However, with a system capable of detecting return light at a high SN ratio, more accurate distance measurement is possible.

In some subjects, capillaries are positioned at a place near a surface of the subject. When such a subject is irradiated with the second irradiation light, the second irradiation light passes through capillaries and reaches a place away from the surface of the subject.

In this optical device, the wavelength band of the second irradiation light includes a wavelength band in which absorption in hemoglobin is large. In this case, the second irradiation light is largely absorbed in capillaries. Thus, even when the second irradiation light reaches a place away from the surface of the subject, the amount of the second irradiation light reaching is extremely small. As a result, the light intensity of the deep layer scattered light is decreased.

Furthermore, the deep layer scattered light directed toward the surface of the subject passes through capillaries. The wavelength band of the deep layer scattered light also includes a wavelength band in which absorption in hemoglobin is large. The deep layer scattered light is therefore largely absorbed in capillaries. As a result, the light intensity of the deep layer scattered light reaching the surface of the subject is further decreased.

As described above, the deep layer scattered light is light causing error information. When the light intensity of the deep layer scattered light is decreased, error information is reduced.

The surface layer scattered light includes scattered light that occurs between a surface of the subject and a capillary and scattered light that occurs in a capillary. The wavelength band of the second irradiation light includes a wavelength band in which absorption in hemoglobin is large. Hence, compared with when the second irradiation light includes a wavelength band of 460 nm or more and 510 nm or less, the light intensity of scattered light that occurs in capillaries is low.

When a capillary is positioned at a place near a surface of the subject, it is possible to consider the position of the capillary to represent the position of the surface of the subject. However, the capillary is not positioned on the surface of the subject. Therefore, if the position of a capillary is too far away from the surface of the subject, the scattered light that occurs in the capillary is light causing error information, like the deep layer scattered light.

As described above, in this optical device, the wavelength band of the second irradiation light includes a wavelength band in which absorption in hemoglobin is large. Hence, the light intensity of the scattered light that occurs in a capillary is lower than the light intensity of the scattered light that occurs between a surface of the subject and a capillary. Even when the scattered light that occurs in a capillary is light causing error information, it is possible to reduce error information.

The light intensity of scattered light that occurs between a surface of the subject and a capillary is low. By using an imager with a high SN ratio as the second imager, it is possible to detect the scattered light that occurs between a surface of the subject and a capillary with a high SN ratio.

As just described, in this optical device, it is possible to measure the distance only with return light from the neighborhood of the surface of the subject. It is therefore possible to accurately measure the distance.

### (Optical Device 2: Sixth Example)

In the optical device of the present embodiment, it is preferable that the second irradiation light be ultraviolet light.

When white light is used as the first irradiation light and light in the visible range is used as the second irradiation light, the wavelength band of the second irradiation light overlaps with the wavelength band of the first irradiation light. As described above, in the optical filter, the return light is separated into the first measurement light and the second measurement light. If the wavelength band of the second irradiation light overlaps with the wavelength band of the first irradiation light, it is difficult to increase the proportion of the second irradiation light included in the second measurement light.

As in an optical device 5 of the present embodiment described later, light in a wavelength band other than the second irradiation light can be eliminated from the second measurement light using a bandpass filter or the like. However, it is not possible to eliminate light in the wavelength band of the second irradiation light from the second measurement light even using a bandpass filter or the like.

In this optical device, ultraviolet light is used as the second irradiation light. When ultraviolet light is used as the second irradiation light, the wavelength band of the second irradiation light does not overlap with the wavelength band of the first irradiation light.

Light emitted from a xenon lamp includes ultraviolet light. When the first irradiation light is wide-band light, for example, light emitted from a xenon lamp, the first irradiation light includes ultraviolet light. Ultraviolet light is unnecessary light for acquisition of image information of the subject in the first imager. Hence, ultraviolet light may be eliminated by an appropriate optical filter before being applied to the subject.

In the optical device of the present example, the ultraviolet region of the first irradiation light is eliminated before the light is incident on the light collecting unit. Therefore, the wavelength band of the second irradiation light does not overlap with the wavelength band of the first irradiation light.

Hence, it is possible to include all the second irradiation light in the second measurement light. When the subject is not a living body, by using ultraviolet light as the second irradiation light, it is possible to brighten both of an optical image of the subject formed on the first imager and an optical image of the subject formed on the second imager. Thus, it is possible to enhance the accuracy of image information and the accuracy of distance information.

When the subject is a living body, the use of ultraviolet light as the second irradiation light may adversely affect the subject. However, it is possible to diminish the adverse effect by properly setting the light intensity and the irradiation time. Thus, even when the subject is a living body, it is possible to enhance the accuracy of image information and the accuracy of the distance information by using ultraviolet light as the second irradiation light.

### (Light Source Unit: Third Example)

As described above, in the optical device, it is possible to use light in various wavelength bands as the second irradiation light. Light in various wavelength bands is generated in the light source unit. Examples of such a light source unit are described below.

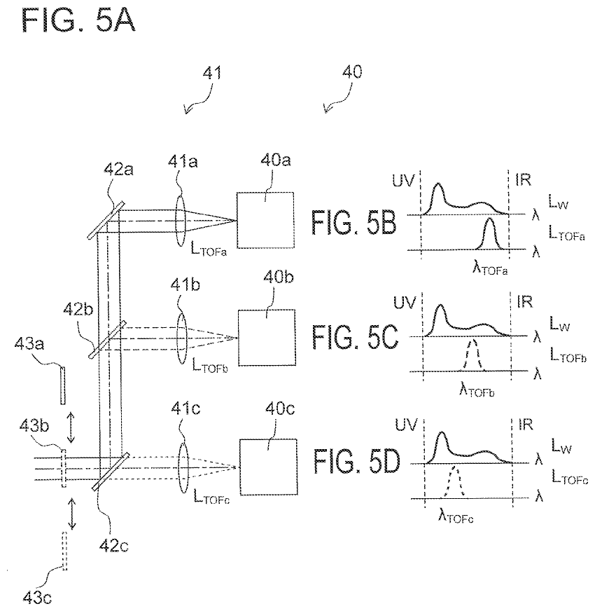

FIG. 5A, FIG. 5B, FIG. 5C, and FIG. 5D are diagrams illustrating the light source unit and wavelengths of irradiation light. FIG. 5A is a diagram illustrating the light source unit. FIG. 5B is a diagram illustrating a first example of the wavelength band of the second irradiation light. FIG. 5C is a diagram illustrating a second example of the wavelength band of the second irradiation light. FIG. 5D is a diagram illustrating a third example of the wavelength band of the second irradiation light.

In FIG. 5A, the first light source is not illustrated. The light source unit includes a second light source unit 40 and a light collecting unit 41. The light source unit further includes a mirror 42a, a dichroic mirror 42b, a dichroic mirror 42c, an optical filter 43a, an optical filter 43b, and an optical filter 43c.

The second light source unit 40 includes a plurality of second light sources. Specifically, the second light source unit 40 includes a second light source 40a, a second light source 40b, and a second light source 40c. The light collecting unit 41 includes a plurality of lenses. Specifically, the light collecting unit 41 includes a lens 41a, a lens 41b, and a lens 41c.

Second irradiation light L_{TOFa} is emitted from the second light source 40a. The second irradiation light L_{TOFa} is light having a peak wavelength λ_{TOFa}. As illustrated in FIG. 5B, the peak wavelength λ_{TOFa} is positioned near the infrared wavelength band IR. The second irradiation light L_{TOFa} is, for example, red light.

Second irradiation light L_{TOFb} is emitted from the second light source 40b. The second irradiation light L_{TOFb} is light having a peak wavelength λ_{TOFb}. As illustrated in FIG. 5C, the peak wavelength λ_{TOFb} is positioned closer to the ultraviolet wavelength band UV than the peak wavelength λ_{TOFa} is. The second irradiation light L_{TOFb} is, for example, green light.

Second irradiation light L_{TOFc} is emitted from the second light source 40c. The second irradiation light L_{TOFc} is light having a peak wavelength λ_{TOFc}. As illustrated in FIG. 5D, the peak wavelength λ_{TOFc} is positioned near the ultraviolet wavelength band UV. The second irradiation light L_{TOFc} is, for example, blue light.

The second irradiation light L_{TOFa} is incident on the lens 41a. The second irradiation light L_{TOFa} is converted into parallel pencil of light by the lens 41a and thereafter emanated from the lens 41a. The second irradiation light L_{TOFa} is incident on the mirror 42a.

The second irradiation light L_{TOFb} is incident on the lens 41b. The second irradiation light L_{TOFb} is converted into parallel pencil of light by the lens 41b and thereafter emanated from the lens 41b. The second irradiation light L_{TOFb} is incident on the dichroic mirror 42b.

The second irradiation light L_{TOFc} is incident on a lens 41c. The second irradiation light L_{TOFc} is converted into parallel pencil of light by the lens 41c and thereafter emanated from the lens 41c. The second irradiation light L_{TOFc} is incident on the dichroic mirror 42c.

The second irradiation light L_{TOFa} is reflected by the mirror 42a and thereafter incident on the dichroic mirror 42b. The dichroic mirror 42b has, for example, the characteristics of transmitting red light and reflecting green light. Thus, the second irradiation light L_{TOFa} is transmitted through the dichroic mirror 42b, and the second irradiation light L_{TOFb} is reflected by the dichroic mirror 42b. The second irradiation light L_{TOFa} and the second irradiation light L_{TOFb} travel toward the dichroic mirror 42c.

The second irradiation light L_{TOFa} and the second irradiation light L_{TOFb} are incident on the dichroic mirror 42c. The dichroic mirror 42c has, for example, the characteristics of transmitting blue light and reflecting red light and green light. Thus, the second irradiation light L_{TOFc} is transmitted through the dichroic mirror 42c, and the second irradiation light L_{TOFa} and the second irradiation light L_{TOFb} are reflected by the dichroic mirror 42c.

The second irradiation light L_{TOFa}, the second irradiation light L_{TOFb}, and the second irradiation light L_{TOFc} travel through the same optical path. As described above, the light source unit includes the optical filter 43a, the optical filter 43b, and the optical filter 43c. These optical filters each can be inserted into the optical path and removed from the optical path.

When the optical filter 43a is inserted into the optical path, the second irradiation light L_{TOFa} is emanated. When the optical filter 43b is inserted into the optical path, the second irradiation light L_{TOFb} is emanated. When the optical filter 43c is inserted into the optical path, the second irradiation light L_{TOFc} is emanated. In this way, it is possible to use light in various wavelength bands as the second irradiation light.

In this case, it is preferable that the first irradiation light and the second irradiation light be multiplexed by a half mirror, the first measurement light and the second measurement light be also demultiplexed by a half mirror, and turning-on of the first light source and turning-on the second light source be alternately performed.

It is possible to use the configuration illustrated in FIG. 5A for the first light source. It is possible to obtain white light if the optical filter 43a, the optical filter 43b, and the optical filter 43c are not used.

### (Optical Device 3 of the Present Embodiment)

In the optical device of the present embodiment, it is preferable that the first light source, the second light source, and the light collecting unit be used for reduction of error information, and an incident angle of the second irradiation light at an incident end face on which the second irradiation light is incident be smaller than an incident angle of the first irradiation light at an incident end face on which the first irradiation light is incident.

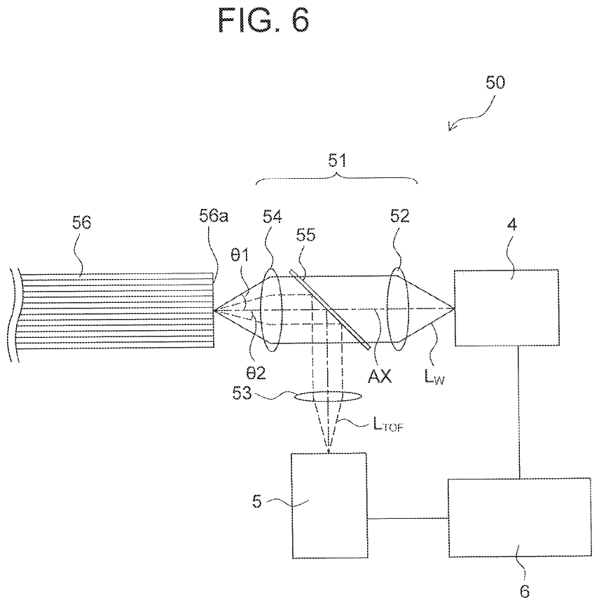

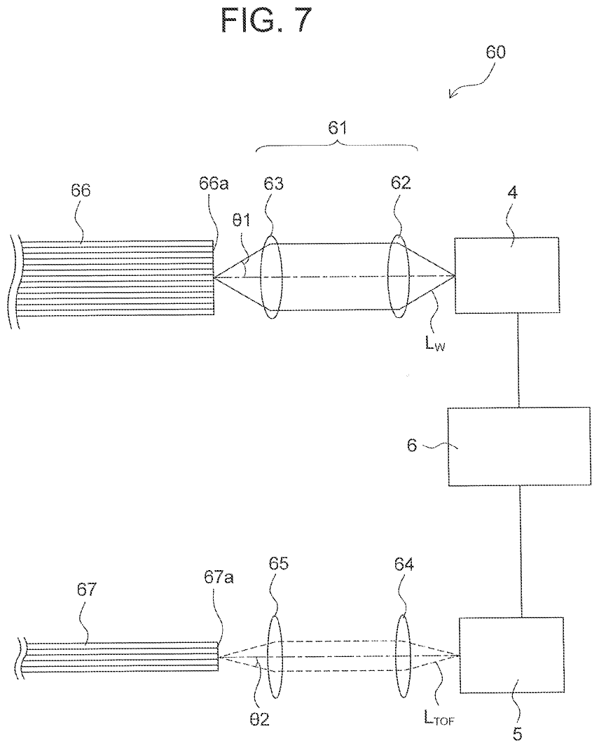

Referring to FIG. 6 and FIG. 7, the light source unit of the optical device will be described. FIG. 6 and FIG. 7 are diagrams illustrating the light source unit. The same component as that in FIG. 1A is denoted by the same numeral and a description thereof is omitted.

It is possible to use a surface light source as a light source in the light source unit of this optical device. The surface light source has a light-emitting surface. It is possible to consider the light-emitting surface to be an assembly of point light sources.

For example, an LED, a xenon lamp, or a halogen lamp is used as the surface light source. An LD is also a surface light source having an emission area with a width of approximately 10 μm and a height of approximately 0.1 μm. It is possible to form a surface light source having a wider area by combining an LD and a fiber. In this case, the wxit end face of the fiber is considered to be a light-emitting surface.

In FIG. 6 and FIG. 7, only light emitted from one point of the light-emitting surface is illustrated for the sake of visibility. One point illustrated in the drawings is a point on the optical axis of an optical system. Light illustrated in FIG. 6 or light illustrated in FIG. 7 is considered to be emitted from various positions of the light-emitting surface.

In FIG. 6 and FIG. 7, an optical system is disposed between the light source and the light guide member. An optical image of the light-emitting surface is formed by the optical system on the incident end face of the light guide member. The optical system need not be strictly disposed to form an optical image of the light-emitting surface. In general, the diameter of the light guide member is smaller than the diameter of the optical system. Hence, the optical system is disposed such that an optical image of the light-emitting surface is substantially formed on the incident end face of the light guide member.

In this optical device, the first light source, the second light source, and the light collecting unit are used for reduction of error information. The first irradiation light and the second irradiation light are emanated from the light collecting unit. The first irradiation light and the second irradiation light are incident on the incident end face of the light guide member. In this case, it is preferable that the incident angle of the second irradiation light at an incident end face on which the second irradiation light is incident be smaller than the incident angle of the first irradiation light at an incident end face on which the first irradiation light is incident.

As illustrated in FIG. 6, a conical pencil of light is incident on an incident end face 56a. The conical pencil of light is formed by a circular pencil of light passing through a lens 54. θ1 and θ2 each are an angle formed between the generatrix of the cone and the optical axis at the intersection of the incident end face 56a and the optical axis AX.

A pencil of light substantially converging on a point other than axial points has substantially the same incident angle. A pencil of light passing through the lens 54 is usually circular, but when it deviates from a circle, it is appropriate to set the longer diameter as a reference.

It is possible to determine θ1 and θ2 by the diameter of a pencil of light passing through the lens 54. When the outer periphery of the pencil of light is clear, it is possible to set the diameter of the outer periphery as the diameter of the pencil of light. When the outer periphery of the pencil of light is not clear, it is possible to set the full width at half maximum as the diameter of the pencil of light. Furthermore, for example, the full width at 20% of the maximum intensity may be used instead of the full width at half maximum.

### (Light Source Unit: Fourth Example)

As illustrated in FIG. 6, a light source unit 50 includes the first light source 4, the second light source 5, the light source control unit 6, and a light collecting unit 51. The light collecting unit 51 includes a lens 52, a lens 53, a lens 54, and a dichroic mirror 55.

In the light source unit 50, two illumination optical paths are formed. The first light source 4 and the lens 52 are disposed on one of the two illumination optical paths, and the second light source 5 and the lens 53 are disposed on the other illumination optical path. The dichroic mirror 55 is disposed at a position where the two illumination optical paths intersect.

First irradiation light L_{W} is emitted from the first light source 4. The first irradiation light L_{W} is white light. The first irradiation light L_{W} passes through the lens 52 and is incident on the dichroic mirror 55. Second irradiation light L_{TOF} is emitted from the second light source 5. The second irradiation light L_{TOF} is narrow-band light. The second irradiation light L_{TOF} passes through the lens 52 and is incident on the dichroic mirror 55.

The first irradiation light L_{W} is transmitted through the dichroic mirror 55. The second irradiation light L_{TOF} is reflected by the dichroic mirror 55. As a result, the first irradiation light L_{W} and the second irradiation light L_{TOF} travel through the same illumination optical path.

The lens 54 is disposed on the same illumination optical path. The first irradiation light L_{W} and the second irradiation light L_{TOF} are collected by the lens 54. The incident end face 56a of a light guide member 56 is disposed at the light collecting position. The first irradiation light L_{W} and the second irradiation light L_{TOF} are incident together on the light guide member 56.

The first irradiation light L_{W} and the second irradiation light L_{TOF} are incident on the incident end face 56a. Thus, the incident end face 56a is an incident end face on which the first irradiation light L_{W} is incident and an incident end face on which the second irradiation light L_{TOF} is incident.

The first irradiation light L_{W} is incident on the incident end face 56a at an angle θ1. The second irradiation light L_{TOF} is incident on the incident end face 56a at an angle θ2. In the light source unit 50, the angle θ2 is smaller than the angle θ1.

The angle θ1 and the angle θ2 both represent an incident angle. Thus, the incident angle of the second irradiation light L_{TOF} at the incident end face 56a is smaller than the incident angle of the first irradiation light L_{W} at the incident end face 56a. When the angle distribution of irradiation light is a distribution that continuously changes like a Gaussian distribution, the angle θ1 and the angle θ2 are the angles at which the light intensity is half the maximum for the light intensity on the axis.

As described above, it is possible to consider the light-emitting surface to be an assembly of point light sources. In the light source unit 50, all the second irradiation light L_{TOF} emitted from each point of the light-emitting surface is incident on the incident end face 56a generally at the incident angle 62 defined above.

It is possible to adjust the incident position of the second irradiation light L_{TOF} at the incident end face 56a by changing the position of the second light source 5.

### (Light Source Unit: Fifth Example)

As illustrated in FIG. 7, a light source unit 60 includes the first light source 4, the second light source 5, the light source control unit 6, and a light collecting unit 61. The light collecting unit 61 includes a lens 62, a lens 63, a lens 64, and a lens 65.

In the light source unit 60, two illumination optical paths are formed. The first light source 4, the lens 62, and the lens 63 are disposed on one of the two illumination optical paths, and the second light source 5, the lens 64, and the lens 65 are disposed on the other illumination optical path.

First irradiation light L_{W} is emitted from the first light source 4. The first irradiation light L_{W} is white light. The first irradiation light L_{W} is collected by the lens 62 and the lens 63. An incident end face 66a of a light guide member 66 is disposed at the light collecting position. The first irradiation light L_{W} is incident on the light guide member 66.

Second irradiation light L_{TOF} is emitted from the second light source 5. The second irradiation light L_{TOF} is narrow-band light. The second irradiation light L_{TOF} is collected by the lens 64 and the lens 65. An incident end face 67a of a light guide member 67 is disposed at the light collecting position. The second irradiation light L_{TOF} is incident on the light guide member 67.

The first irradiation light L_{W} is incident on the incident end face 66a. Thus, the incident end face 66a is an incident end face on which the first irradiation light L_{W} is incident. The second irradiation light L_{TOF} is incident on the incident end face 67a. Thus, the incident end face 67a is an incident end face on which the second irradiation light L_{TOF} is incident.

The first irradiation light L_{W} is incident on the incident end face 66a at an angle θ1. The second irradiation light L_{TOF} is incident on the incident end face 67a at an angle θ2. In the light source unit 60, the angle θ2 is smaller than the angle θ1.

The angle θ1 and the angle θ2 both represent an incident angle. Thus, the incident angle of the second irradiation light L_{TOF} at the incident end face 67a is smaller than the incident angle of the first irradiation light L_{W} at the incident end face 66a.

As described above, it is possible to consider the light-emitting surface to be an assembly of point light sources. In the light source unit 60, all the second irradiation light L_{TOF} emitted from each point of the light-emitting surface is incident on the incident end face 67a generally at the angle θ2.

In the light source unit 50 and the light source unit 60, pulsed light is used as the second irradiation light L_{TOF}. In the pulsed light, the pulse shape is rectangular. In order to perform accurate measurement, it is desirable that the pulse shape do not change.

In the light guide member 56, there are various propagation modes. When the propagation modes vary, the propagation time of pulsed light also varies. Pulsed light is emanated from the light guide member 56 in a state in which pulsed light propagating in various propagation modes is combined. Hence, even when the pulse shape is rectangular at a time of being incident on the light guide member 56, the pulse shape of pulsed light emanated from the light guide member 56 becomes non-rectangular. That is, in the light guide member 56, the pulse shape changes while the pulsed light propagates through the light guide member 56. This is applicable to the light guide member 67.

In the optical device of the present embodiment, the angle θ2 is smaller than the angle θ1. Hence, it is possible to reduce the number of propagation modes. As a result, it is possible to diminish the change in pulse shape in the second irradiation light L_{TOF}.

In the optical device of the present embodiment, it is possible to minimize the change in pulse shape of the second irradiation light emanated from one exit end face. This is effective in improvement in accuracy in distance measurement.

When the second irradiation light is emanated from one exit end face, the effect of improving the accuracy in distance measurement is further increased for the reason explained in an optical device 11 described later.

As described above, the change in pulse shape means that error information is added to distance information. In the optical device of the present embodiment, it is possible to reduce error information because it is possible to diminish the change in pulse shape.

### (Optical Device 3: Seventh Example)

In the optical device of the present embodiment, it is preferable that the incident angle of the second irradiation light be 5.7° or less.

It is possible to use the optical device in a flexible endoscope. In this case, it is possible to use the light source unit 50 or the light source unit 60 in a flexible endoscope.

In a flexible endoscope, a surface of the upper digestive tract, for example, a surface of the stomach is observed at a distance of about 5 cm. In observation, an image acquired by the imager is displayed on a monitor. During observation, a lesion area may be detected.

If the size of the lesion area can be measured within an error of about 10%, it is possible to use the measurement result for a definitive diagnosis of the lesion area. In order to measure the size of the lesion area within an error of about 10%, the distance from the endoscope to the surface of the subject must be measured within an error of about 10%.

As illustrated in FIG. 6, the second irradiation light L_{TOF} is incident in a collected state on the incident end face 56a. Thus, the second irradiation light L_{TOF} is incident on the incident end face 56a at various angles from 0° to θ2.

The second irradiation light L_{TOF} incident on the light guide member 56 at an angle of θ2 propagates through the light guide member 56 while being repeatedly reflected by the light guide member 56. The second irradiation light L_{TOF} incident on the light guide member 56 at an angle of 0° propagates through the light guide member 56 without being reflected by the light guide member 56. Hence, the second irradiation light L_{TOF} incident on the light guide member 56 at the angle θ2 reaches the exit end face of the light guide member 56 later than the second irradiation light L_{TOF} incident on the light guide member 56 at an angle of 0°.

For example, the edge portion in the pulse shape of light with light intensity temporally modulated at 100 MHz becomes dull while the light propagates through the light guide member. Furthermore, a phase lag occurs. In this case, since the pulse shape is changed, the pulse shape becomes non-rectangular. The change in pulse shape means that error information is added to distance information.

It is possible to obtain an error d by the following Expressions (a), (b), and (c):

$$\begin{matrix}d = n \times df & ( a ) \\ df = ( 1 - cos φ ) \times L & ( b ) \\ sin θ / sin φ = n & ( c )\end{matrix}$$

where

n is a refractive index of the light guide member,

df is a delay that occurs between first light and second light in the interior of the light guide member,

θ is an incident angle of the first light,

L is an entire length of the light guide member,

the first light is light incident on the light guide member at an angle θ, and

the second light is light incident on the light guide member at an angle of 0°.

df is the delay that occurs between the first light and the second light in the interior of the light guide member. Thus, the error d is a delay that occurs between the first light and the second light outside the light guide member.

It is possible to determine the angle θ by measuring light incident on the light guide member with a goniophotometer. In the goniophotometer, it is possible to determine a light distribution. It is possible to determine the angle θ by half width at half maximum in a light distribution.

As can be understood from Expressions (a), (b), and (c), the larger the incident angle of the first light is, or the longer the entire length of the light guide member is, the greater the error d is.

If the surface of the subject has a step having a length of dL, a time delay equivalent to 2×dL occurs at the step. In a flexible endoscope, distance measurement is performed at a distance of about 5 cm. In this case, in order to keep an error within 10% for the distance of 5 cm, it is necessary to set d to 10 mm or less when the error is 5 mm.

It is possible to use the optical device 1 in a flexible endoscope. In such a flexible endoscope, when the light source unit 2 is disposed at a position away from the body unit 3, the value of L is 3000 mm. When n=1.5 and d=10 mm, θ≈5.7°.

When the optical device 1 is used in a flexible endoscope, it is possible to use the light source unit 50 as the light source unit of the flexible endoscope. In this case, the incident angle of the second irradiation light L_{TOF} at the incident end face 56a is preferably set to 5.7° or less. By doing so, it is possible to diminish the change in pulse shape. As a result, it is possible to reduce error information.

The light source unit 60 may be used as the light source unit of the flexible endoscope. In this case, the incident angle of the second irradiation light L_{TOF} at the incident end face 67a is preferably set to 5.7° or less.

### (Optical Device 3: Eighth Example)

In the optical device of the present embodiment, it is preferable that the incident angle of the second irradiation light be 2.5° or less.

As described above, it is possible to use the optical device in a flexible endoscope. In a flexible endoscope, in the case of close observation, a surface of the upper digestive tract is sometimes observed at a distance of about 1 cm. In this case, in order to keep an error within 10% or less, it is necessary to set d to 0.2 mm or less.

It is possible to use the optical device 1 in a flexible endoscope. In such a flexible endoscope, when the light source unit 2 is disposed at a position away from the body unit 3, the value of L is 3000 mm. When n=1.5 and d=0.2 mm, θ≈2.5°.

As described above, it is possible to use the light source unit 50 as the light source unit of the flexible endoscope. In this case, the incident angle of the second irradiation light L_{TOF} at the incident end face 56a is preferably set to 2.5° or less. By doing so, it is possible to diminish the change in pulse shape. As a result, it is possible to reduce error information.

The light source unit 60 may be used as the light source unit of the flexible endoscope. In this case, the incident angle of the second irradiation light L_{TOF} at the incident end face 67a is preferably set to 2.5° or less.

### (Optical Device 4 of the Present Embodiment)

In the optical device of the present embodiment, it is preferable that the light causing error information be predetermined light included in the first irradiation light, the predetermined light be light including the same wavelength band as the wavelength band of the second irradiation light, and predetermined light included in the second measurement light be reduced.

In the optical device of the present embodiment, it is more preferable that the light causing error information be predetermined light included in the first irradiation light, the predetermined light be light in the same wavelength band as the wavelength band of the second irradiation light, and predetermined light included in the second measurement light be reduced.

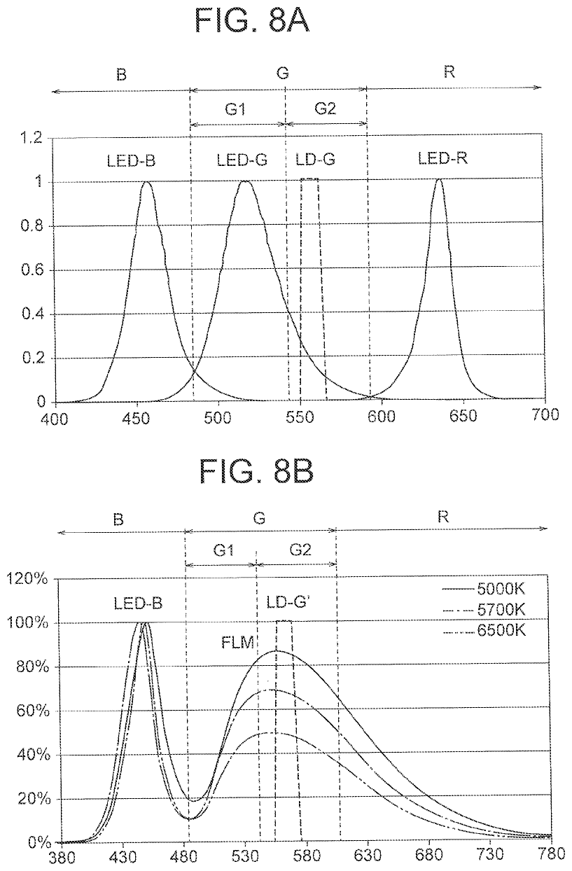

FIG. 8A and FIG. 8B are diagrams illustrating a spectral distribution of the first irradiation light and a spectral distribution of the second irradiation light. FIG. 8A is a diagram illustrating a first example of the spectral distributions. FIG. 8B is a diagram illustrating a second example of the spectral distributions. The distribution curve of the first irradiation light is depicted by a solid line, and the distribution curve of the second irradiation light is depicted by a broken line.

In the optical device, the first light source and the second light source are used. The first light source is a light source for image acquisition. The second light source is a light source for TOF. Here, a white LED is used as the first light source, and a monochrome LD is used as the second light source.

The first irradiation light is emitted from the first light source. The second irradiation light is emitted from the second light source. Thus, the spectral distributions illustrated in FIG. 8A and the spectral distributions illustrated in FIG. 8B represent spectral distributions of light emitted from the white LED and spectral distributions of light emitted from the monochrome LD.

### (Spectral Distribution: First Example)

A plurality of LEDs are used for the white LED. The LEDs include, for example, an LED-B, an LED-G, and an LED-R. The LED-B is an LED emitting blue light, the LED-G is an LED emitting green light, and the LED-R is an LED emitting red light.

For example, an LD-G is used for the monochrome LD. The LD-G is an LD emitting green light.

As illustrated in FIG. 8A, in the LED-B, a peak of light intensity is positioned in a wavelength band B. In the LED-G, a peak of light intensity is positioned in a wavelength band G. In the LED-R, a peak of light intensity is positioned in a wavelength band R. In the LD-G, a peak of light intensity is positioned in a wavelength band G2.

On the long wavelength side of the distribution curve of the LED-B and the short wavelength side of the distribution curve of the LED-G, these curves intersect each other before the light intensity becomes zero. On the long wavelength side of the distribution curve of the LED-G and the short wavelength side of the distribution curve of the LED-R, these curves intersect each other before the light intensity becomes zero.

In the white LED illustrated in FIG. 8A, the light intensity is not zero in any of the wavelength band B, the wavelength band G, and the wavelength band R. Thus, the first irradiation light is white light having continuous spectra.

### (Spectral Distribution: Second Example)

One LED and one phosphor are used for the white LED. This LED is, for example, the LED-B described above. A phosphor FLM is, for example, a phosphor emitting yellow fluorescence.

For example, an LD-G′ is used for the monochrome LD. The LD-G′ is an LED emitting green light.

As illustrated in FIG. 8B, in the LED-B, a peak of light intensity is positioned in a wavelength band B. In the phosphor FLM, a peak of light intensity is positioned in a wavelength band G. In the LD-G′, a peak of light intensity is positioned in a wavelength band G2.

In the spectral distribution of the white LED illustrated in FIG. 8B, the light intensity is not zero in any of the wavelength band B, the wavelength band G, and the wavelength band R. Thus, the first irradiation light is white light having continuous spectra.

As illustrated in FIG. 8A and FIG. 8B, the wavelength band of the white LED is formed of the wavelength band B, the wavelength band G, and the wavelength band R. The wavelength band of the monochrome LD is included in the wavelength band G2.

The wavelength band of the white LED represents the wavelength band of the first irradiation light, and the wavelength band of the monochrome LD represents the wavelength band of the second irradiation light. Thus, the first irradiation light includes light (hereinafter referred to as “predetermined light”) including the same wavelength band as the wavelength band of the second irradiation light.

The influence of the predetermined light on distance measurement will be described. As illustrated in FIG. 8A and FIG. 8B, the wavelength band of the second irradiation light is distributed in the wavelength band G2. Thus, the predetermined light includes part of light of the LED-G and light of the LD-G.

### (Ideal Dichroic Mirror)

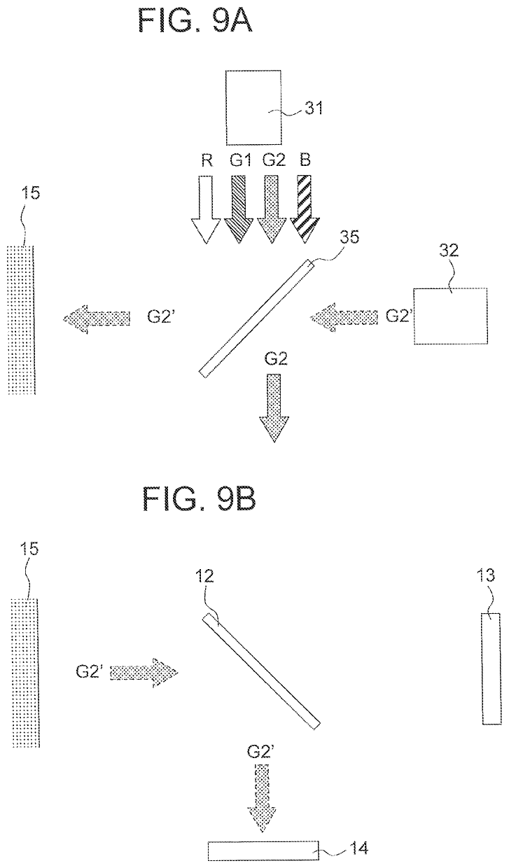

FIG. 9A and FIG. 9B are diagrams illustrating irradiation light and measurement light. FIG. 9A is a diagram illustrating irradiation light. FIG. 9B is a diagram illustrating measurement light. The same component as that in FIG. 1A and FIG. 3A is denoted by the same numeral and a description thereof is omitted.

First irradiation light R, first irradiation light G1, predetermined light G2, and first irradiation light B are depicted by solid arrows. Second irradiation light G2′ is depicted by dotted arrows.

The first light source 31 and the second light source 32 are simultaneously turned on. However, in order to give explanation focusing on the light in the wavelength band G2, the first irradiation light R, the first irradiation light G1, and the first irradiation light B are partly omitted in FIG. 9A and FIG. 9B.

In FIG. 9A and FIG. 9B, ideal dichroic mirrors are used for the dichroic mirror 35 and the optical filter 12. In an ideal dichroic mirror, the transmittance for light in the wavelength band G2 is 100%, or the reflectivity for light in the wavelength band G2 is 100%.

As illustrated in FIG. 9A, the first irradiation light R, the first irradiation light G1, the predetermined light G2, and the first irradiation light B are emitted from the first light source 31. The second irradiation light G2′ is emitted from the second light source 32. The wavelength band of the second irradiation light G2′ matches with a part of the wavelength band of the predetermined light G2.

The first irradiation light R is light in the wavelength band R. The first irradiation light G1 is light in the wavelength band G1. The predetermined light G2 is light in the wavelength band G2. The first irradiation light B is light in the wavelength band B. The second irradiation light G2′ is light in the same wavelength band as a part of the wavelength band G2. Each wavelength band is illustrated in, for example, FIG. 8A or FIG. 8B.

The first irradiation light R, the first irradiation light G1, the predetermined light G2, the first irradiation light B, and the second irradiation light G2′ are incident on the dichroic mirror 35. In FIG. 9A, a dichroic mirror having a transmittance of 100% for light in the wavelength band G2 is used as the dichroic mirror 35.

The predetermined light G2 is transmitted through the dichroic mirror 35 and therefore not reflected by the dichroic mirror 35. The second irradiation light G2′ is transmitted through the dichroic mirror 35. As a result, the subject 15 is irradiated with the second irradiation light G2′.

As illustrated in FIG. 9B, the second irradiation light G2′ returns from the subject 15. The second irradiation light G2′ is incident on the optical filter 12.

A dichroic mirror having a reflectivity of 100% for light in the wavelength band G2′ is used as the optical filter 12. Hence, the second irradiation light G2′ is reflected by the optical filter 12. As a result, only the second irradiation light G2′ is incident as the second measurement light on the second imager 14.

In actuality, it is difficult to fabricate an ideal dichroic mirror. Thus, a practical dichroic mirror is used.

### (Practical Dichroic Mirror)

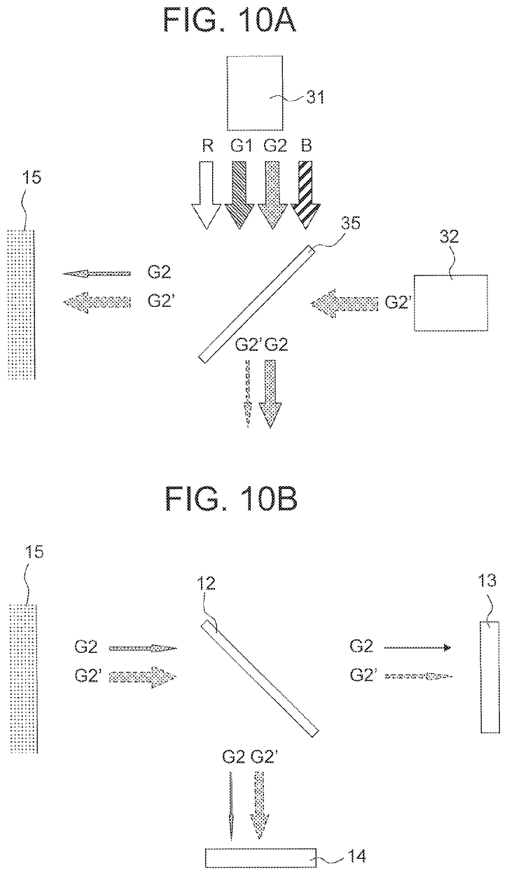

FIG. 10A and FIG. 10B are diagrams illustrating irradiation light and measurement light. FIG. 10A is a diagram illustrating irradiation light. FIG. 10B is a diagram illustrating measurement light. The same component as that in FIG. 9A and FIG. 9B is denoted by the same numeral and a description thereof is omitted.

The first light source 31 and the second light source 32 are simultaneously turned on. However, in order to give explanation focusing on light in the wavelength band G2, the first irradiation light R, the first irradiation light G1, and the first irradiation light B are partly omitted also in FIG. 10A and FIG. 10B.

In FIG. 10A and FIG. 10B, practical dichroic mirrors are used for the dichroic mirror 35 and the optical filter 12. In a practical dichroic mirror, the transmittance for light in the wavelength band G2 is less than 100%, or the reflectivity for light in the wavelength band G2 is less than 100%.

As illustrated in FIG. 10A, the first irradiation light R, the first irradiation light G1, the predetermined light G2, the first irradiation light B, and the second irradiation light G2′ are incident on the dichroic mirror 35. A dichroic mirror having a transmittance of less than 100% for light in the wavelength band G2 is used as the dichroic mirror 35.

Hence, the predetermined light G2 is split into light reflected by the dichroic mirror 35 and light transmitted through the dichroic mirror 35. The second irradiation light G2′ is also split into light transmitted through the dichroic mirror 35 and light reflected by the dichroic mirror 35. As a result, the subject 15 is irradiated with the predetermined light G2 and the second irradiation light G2′.

As illustrated in FIG. 10B, the predetermined light G2 and the second irradiation light G2′ return from the subject 15. The predetermined light G2 and the second irradiation light G2′ are incident on the optical filter 12.

A dichroic mirror having a reflectivity of less than 100% for light in the wavelength band G2 is used as the optical filter 12. Hence, the predetermined light G2 and the second irradiation light G2′ are split into light reflected by the optical filter 12 and light transmitted through the optical filter 12. As a result, the predetermined light G2 and the second measurement light G2′ are incident as the second measurement light on the second imager 14.

As described above, in an ideal dichroic mirror, the predetermined light G2 is not reflected by the dichroic mirror 35. Hence, the light irradiating the subject 15 does not include the predetermined light G2. That is, the subject 15 is irradiated with irradiation light that does not include light causing error information.

In this case, only the second irradiation light G2′ is incident as the second measurement light on the second imager 14. The second irradiation light G2′ is light having distance information. Thus, it is possible to perform distance measurement with high accuracy.

By contrast, in a practical dichroic mirror, the predetermined light G2 is reflected by the dichroic mirror 35. Hence, the light irradiating the subject 15 includes the predetermined light G2.

The predetermined light G2 is light included in the first irradiation light. The first irradiation light does not have distance information and therefore is light causing error information. Thus, the predetermined light G2 is light causing error information. In the practical dichroic mirror, the subject 15 is irradiated with irradiation light including light causing error information.

In this case, not only the second irradiation light G2′ but also the predetermined light G2 is incident as the second measurement light on the second imager 14. The second irradiation light G2′ is light having distance information, and the predetermined light G2 is light causing error information.

The wavelength band of a part of the wavelength band of the predetermined light G2 is the same as the wavelength band of the second irradiation light G2′. Hence, it is not possible to separate partial light of the predetermined light G2 from the second irradiation light G2′. That is, it is not possible to separate light causing error information from light including distance information. Thus, it is difficult to perform distance measurement with high accuracy.

However, in the optical device, the predetermined light included in the second measurement light is reduced. Thus, even when the practical dichroic mirror is used, it is possible to perform distance measurement with high accuracy.

### (Optical Device 4: Ninth Example)

In the optical device of the present embodiment, it is preferable that the configuration for reducing the predetermined light included in the second measurement light be a configuration in which the wavelength band of the first irradiation light is wider than the wavelength band of the second irradiation light, the first irradiation light has a plurality of peak wavelengths at which light intensity is maximal, the second irradiation light has one peak wavelength at which light intensity is maximal, and the peak wavelength of the second irradiation light is positioned between adjacent two peak wavelengths of the first irradiation light.

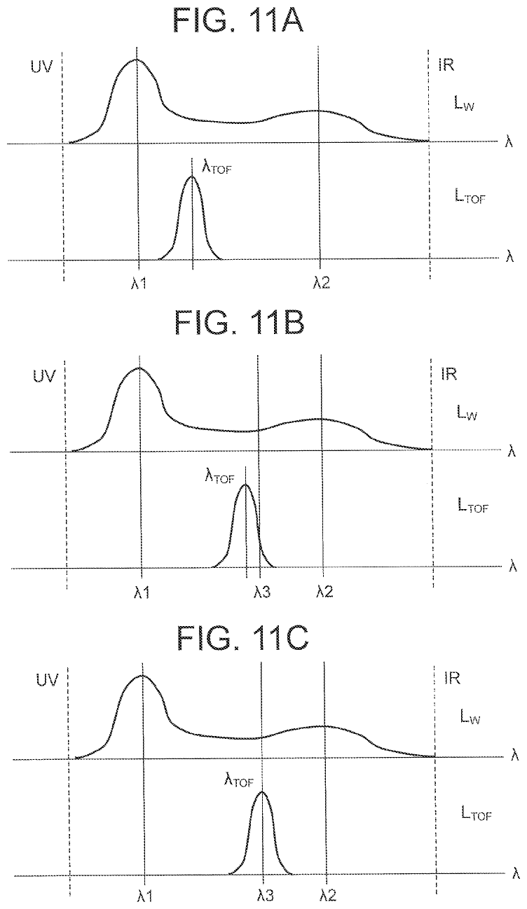

FIG. 11A, FIG. 11B, and FIG. 11C are diagrams illustrating the wavelength band of the first irradiation light and the wavelength band of the second irradiation light. FIG. 11A is a diagram illustrating a first example of the spectral distribution of the second irradiation light. FIG. 11B is a diagram illustrating a second example of the spectral distribution of the second irradiation light. FIG. 11C is a diagram illustrating a third example of the spectral distribution of the second irradiation light.

It is possible to use white light as the first irradiation light L_{W}. It is possible to use narrow-band light as the second irradiation light L_{TOF}. In this case, as illustrated in FIG. 11A, the wavelength band of the first irradiation light L_{W} is wider than the wavelength band of the second irradiation light L_{TOF}.

It is possible to use a white LED or a white LD as the light source of the first irradiation light L_{W}. In a white LED, as illustrated in FIG. 8A and FIG. 8B, a plurality of peak wavelengths at which light intensity is maximal often exist.

On the other hand, the wavelength band of the second irradiation light L_{TOF} need not be wide. Typically, a monochrome LED or a monochrome LD, for example, is used as the light source of the second irradiation light L_{TOF}. In such a light source, one peak wavelength at which light intensity is maximum often exists.

In FIG. 11A, a peak wavelength λ1, a peak wavelength λ2, and a peak λ_{TOF} are illustrated. The peak wavelength λ1 and the peak wavelength λ2 are peak wavelengths in the first irradiation light L_{W}. The peak wavelength λ_{TOF} is the peak wavelength in the second irradiation light L_{TOF}. The wavelength band of the first irradiation light L_{W} includes the same wavelength band as the wavelength band of the second irradiation light L_{TOF}.

The second irradiation light L_{TOF} is light having distance information. On the other hand, the first irradiation light L_{W} does not have distance information and therefore is light causing error information. When the second measurement light includes the predetermined light and the second irradiation light L_{TOF}, it is not possible to separate the predetermined light from the second irradiation light L_{TOF}.

It is possible to consider the predetermined light to be noise light. If the predetermined light has high light intensity, the SN ratio of the second measurement light deteriorates. As a result, it is not possible to obtain distance information precisely.

In the optical device of the present embodiment, the peak λ_{TOF} of the second irradiation light L_{TOF} is positioned between the peak wavelength λ1 and the peak wavelength λ2. The peak wavelength λ1 and the peak wavelength λ2 are adjacent two peak wavelengths.

Between the peak wavelength λ1 and the peak wavelength λ2, the light intensity of the first irradiation light L_{W} is low. Hence, by positioning the peak wavelength λ_{TOF} between the peak wavelength λ1 and the peak wavelength λ2, it is possible to decrease the light intensity of the predetermined light. That is, it is possible to reduce the predetermined light included in the second measurement light.

As just described, it is possible to reduce error information by using the wavelength band including the peak λ_{TOF} and in which the light intensity of the predetermined light is low, as the wavelength band of the second irradiation light L_{TOF}. As a result, it is possible to obtain distance information.

The peak wavelength λ1 and the peak wavelength λ2 are positioned in a wavelength band on the shorter wavelength side than the infrared wavelength band. Therefore, in the optical device of the present embodiment, it is possible to use short-wavelength light as the second irradiation light L_{TOF}. As a result, it is possible to reduce error information.

In the first irradiation light L_{W}, a plurality of peak wavelengths are included in the visible range. Hence, the short-wavelength light is also light in the visible range. When the subject is a living body, the second irradiation light L_{TOF} may adversely affect the subject if it is light having a wavelength shorter than the visible range. Since the short-wavelength light used as the second irradiation light L_{TOF} is light in the visible range, it is possible to accurately measure the distance without adversely affecting the subject even when the subject is a living body.

### (Optical Device 4: Tenth Example)

In the optical device of the present embodiment, it is preferable that a bottom wavelength at which light intensity is minimal be included between adjacent two peak wavelengths of the first irradiation light, and the wavelength band of the second irradiation light include the bottom wavelength.

As illustrated in FIG. 11B, in the first irradiation light L_{W}, a bottom wavelength λ3 is positioned between the peak wavelength λ1 and the peak wavelength λ2. The peak λ_{TOF} of the second irradiation light L_{TOF} is positioned near the bottom wavelength λ3. Hence, the wavelength band of the second irradiation light L_{TOF} includes the bottom wavelength λ3.

In the bottom wavelength λ3, the light intensity of the first irradiation light L_{W} is extremely low. Hence, by positioning the peak λ_{TOF} of the second irradiation light L_{TOF} near the bottom wavelength λ3, it is possible to further diminish the light intensity of the predetermined light. That is, it is possible to further reduce the predetermined light included in the second measurement light.

Hence, it is possible to further reduce error information. As a result, it is possible to obtain distance information more precisely.

In this optical device, it is possible to use short-wavelength light as the second irradiation light L_{TOF}. As a result, it is possible to reduce error information. Furthermore, since the short-wavelength light used as the second irradiation light L_{TOF} is light in the visible range, it is possible to accurately measure the distance without adversely affecting the subject even when the subject is a living body.

### (Optical Device 4: Eleventh Example)

In the optical device of the present embodiment, it is preferable that the peak wavelength of the second irradiation light match with the bottom wavelength.

As illustrated in FIG. 11C, in the first irradiation light L_{W}, the bottom wavelength λ3 is positioned between the peak wavelength λ1 and the peak wavelength λ2. The peak λ_{TOF} of the second irradiation light L_{TOF} matches with the bottom wavelength λ3. Hence, the wavelength band of the second irradiation light L_{TOF} includes the bottom wavelength λ3.

In the bottom wavelength λ3, the light intensity of the first irradiation light L_{W} is extremely low. Hence, by matching the peak λ_{TOF} of the second irradiation light L_{TOF} with the bottom wavelength λ3, it is possible to further diminish the light intensity of the predetermined light. That is, it is possible to further reduce the predetermined light included in the second measurement light.

Hence, it is possible to further reduce error information. As a result, it is possible to obtain distance information more precisely.

In this optical device, it is possible to use short-wavelength light as the second irradiation light L_{TOF}. As a result, it is possible to reduce error information. Furthermore, since the short-wavelength light used as the second irradiation light L_{TOF} is light in the visible range, it is possible to accurately measure the distance without adversely affecting the subject even when the subject is a living body.

### (Optical Device 4: Twelfth Example)

In the optical device of the present embodiment, it is preferable that the first irradiation light do not include the predetermined light.

By doing so, the light irradiating the subject does not include the predetermined light. That is, the subject is irradiated with irradiation light that does not include light causing error information.

In this case, only the second irradiation light is incident on the second imager as the second measurement light. The second irradiation light is light having distance information. Thus, it is possible to perform distance measurement with high accuracy.

By narrowing the wavelength band of the predetermined light, it is possible to acquire an image with white light illumination while performing distance measurement.

### (Optical Device 5 of the Present Embodiment)

In the optical device of the present embodiment, it is preferable that the optical system include a bandpass filter, the bandpass filter have spectral characteristics of transmitting light including the same wavelength band as the wavelength band of the second irradiation light and having a transmission band narrower than the wavelength band of the first irradiation light, and the second measurement light be light transmitted through the bandpass filter.

In the optical device of the present embodiment, it is more preferable that the optical system include a bandpass filter, the bandpass filter have spectral characteristics of transmitting only light in the same wavelength band as the wavelength band of the second irradiation light, and the second measurement light be light transmitted through the bandpass filter.

As described above, the predetermined light influences distance measurement in some cases. Light other than the predetermined light (hereinafter referred to as “remaining light”) also influences distance measurement in some cases. The influence of the remaining light on distance measurement will be described.

In the explanation above, only the light in the wavelength band G2 is the predetermined light. Thus, the remaining light is light in the wavelength band B, light in the wavelength band G1, and light in the wavelength band R.

### (Ideal Dichroic Mirror)

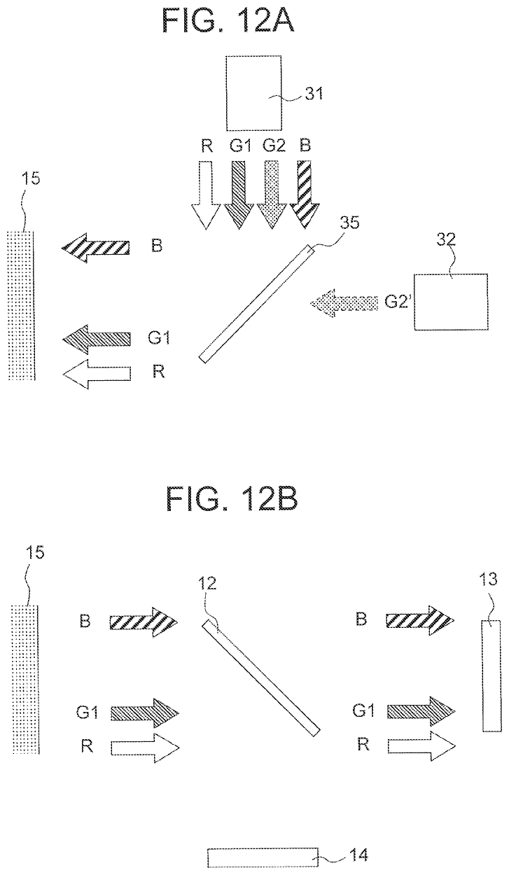

FIG. 12A and FIG. 12B are diagrams illustrating irradiation light and measurement light. FIG. 12A is a diagram illustrating irradiation light. FIG. 12B is a diagram illustrating measurement light. The same component as that in FIG. 9A and FIG. 9B is denoted by the same numeral and a description thereof is omitted.

The first light source 31 and the second light source 32 are simultaneously turned on. However, in order to give explanation focusing on the remaining light, the predetermined light G2 and the second irradiation light G2′ are partly omitted in FIG. 12A and FIG. 12B.

In FIG. 12A and FIG. 12B, ideal dichroic mirrors are used for the dichroic mirror 35 and the optical filter 12. In an ideal dichroic mirror, the transmittance for the remaining light is 100%, or the reflectivity for the remaining light is 100%.

As illustrated in FIG. 12A, the first irradiation light R, the first irradiation light G1, the predetermined light G2, and the first irradiation light B are emitted from the first light source 31. The second irradiation light G2′ is emitted from the second light source 32.

The first irradiation light R, the first irradiation light G1, the predetermined light G2, the first irradiation light B, and the second irradiation light G2′ are incident on the dichroic mirror 35. In FIG. 12A, a dichroic mirror having a reflectivity of 100% for the remaining light is used as the dichroic mirror 35.

The first irradiation light R, the first irradiation light G1, and the first irradiation light B are reflected by the dichroic mirror 35 and therefore not transmitted through the dichroic mirror 35. As a result, the subject 15 is irradiated with the first irradiation light R, the first irradiation light G1, and the first irradiation light B.

As illustrated in FIG. 12B, the first irradiation light R, the first irradiation light G1, and the first irradiation light B return from the subject 15. The first irradiation light R, the first irradiation light G1, and the first irradiation light B are incident on the optical filter 12.

A dichroic mirror having a transmittance of 100% for the remaining light is used as the optical filter 12. In this case, the first irradiation light R, the first irradiation light G1, and the first irradiation light B are transmitted through the optical filter 12 and therefore not reflected by the optical filter 12. As a result, the first irradiation light R, the first irradiation light G1, and the first irradiation light B are not incident as the second measurement light on the second imager 14.

As described above, in actuality, it is difficult to fabricate an ideal dichroic mirror. Thus, a practical dichroic mirror is used.

### (Practical Dichroic Mirror)

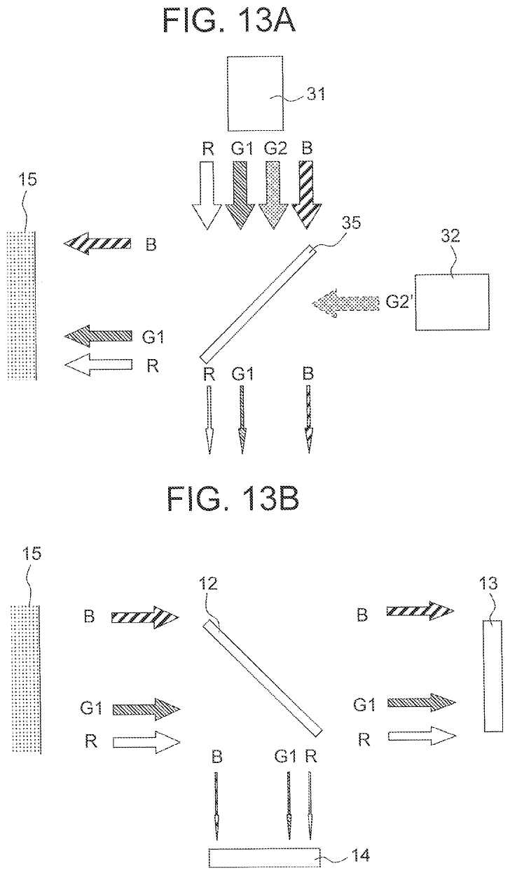

FIG. 13A and FIG. 13B are diagrams illustrating irradiation light and measurement light. FIG. 13A is a diagram illustrating irradiation light. FIG. 13B is a diagram illustrating measurement light. The same component as that in FIG. 9A and FIG. 9B is denoted by the same numeral and a description thereof is omitted.

The first light source 31 and the second light source 32 are simultaneously turned on. However, in order to give explanation focusing on the remaining light, the predetermined light G2 and the second irradiation light G2′ are partly omitted also in FIG. 13A and FIG. 13B.

In FIG. 13A and FIG. 13B, practical dichroic mirrors are used for the dichroic mirror 35 and the optical filter 12. In a practical dichroic mirror, the transmittance for the remaining light is less than 100%, or the reflectivity for the remaining light is less than 100%.

As illustrated in FIG. 13A, the first irradiation light R, the first irradiation light G1, the predetermined light G2, the first irradiation light B, and the second irradiation light G2′ are incident on the dichroic mirror 35. A dichroic mirror having a transmittance of less than 100% for the remaining light is used as the dichroic mirror 35.

Hence, the first irradiation light R, the first irradiation light G1, and the first irradiation light B are split into light reflected by the dichroic mirror 35 and light transmitted through the dichroic mirror 35. As a result, the subject 15 is irradiated with the first irradiation light R, the first irradiation light G1, and the first irradiation light B.

As illustrated in FIG. 13B, the first irradiation light R, the first irradiation light G1, and the first irradiation light B return from the subject 15. The first irradiation light R, the first irradiation light G1, and the first irradiation light B are incident on the optical filter 12.

A dichroic mirror having a transmittance of less than 100% for the remaining light is used as the optical filter 12. Hence, the first irradiation light R, the first irradiation light G1, and the first irradiation light B are split into light transmitted through the optical filter 12 and light reflected by the optical filter 12. As a result, the first irradiation light R, the first irradiation light G1, and the first irradiation light B are incident as the second measurement light on the second imager 14.

As described above, in the ideal dichroic mirror, the first irradiation light R, the first irradiation light G1, and the first irradiation light B are not reflected by the optical filter 12. In this case, the first irradiation light R, the first irradiation light G1, and the first irradiation light B are not incident as the second measurement light on the second imager 14.

The first irradiation light R, the first irradiation light G1, and the first irradiation light B are light included in the first irradiation light. The first irradiation light does not have distance information and therefore is light causing error information. Thus, the first irradiation light R, the first irradiation light G1, and the first irradiation light B are light causing error information.

In an ideal dichroic mirror, light causing error information is not incident on the second imager 14. Thus, it is possible to perform distance measurement with high accuracy.

By contrast, in a practical dichroic mirror, the first irradiation light R, the first irradiation light G1, and the first irradiation light B are reflected by the optical filter 12. Hence, light causing error information is incident on the second imager 14. Thus, it is difficult to perform distance measurement with high accuracy.

In order to perform distance measurement with high accuracy, incidence of the remaining light on the second imager may be blocked. As described above, in the optical device, the optical system includes a bandpass filter. It is possible to block incidence of the remaining light on the second imager by the bandpass filter.

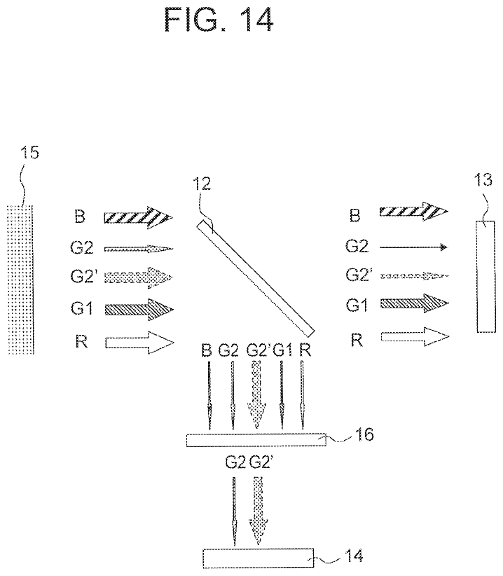

FIG. 14 is a diagram illustrating measurement light. The same component as that in FIG. 13B is denoted by the same numeral and a description thereof is omitted.

In FIG. 14, the first light source 31 and the second light source 32 are simultaneously turned on. Furthermore, practical dichroic mirrors are used for the dichroic mirror 35 and the optical filter 12. Hence, the subject 15 is irradiated with the first irradiation light R, the first irradiation light G1, the predetermined light G2, the first irradiation light B, and the second irradiation light G2′.

The first irradiation light R, the first irradiation light G1, the predetermined light G2, the first irradiation light B, and the second irradiation light G2′ return from the subject 15. These rays of light are incident on the optical filter 12 and split into light reflected by the optical filter 12 and light transmitted through the optical filter 12. As a result, the first irradiation light R, the first irradiation light G1, the predetermined light G2, the first irradiation light B, and the second irradiation light G2′ are directed toward the second imager 14.

In this optical system, a bandpass filter 16 is disposed between the optical filter 12 and the second imager 14. The first irradiation light R, the first irradiation light G1, the predetermined light G2, the first irradiation light B, and the second irradiation light G2′ are incident on the bandpass filter 16.

The bandpass filter 16 has spectral characteristics of transmitting only light in the same wavelength band as the wavelength band of the predetermined light G2. Hence, the first irradiation light R, the first irradiation light G1, and the first irradiation light B are reflected by the bandpass filter 16.

As a result, it is possible to allow only the second irradiation light G2′ and the predetermined light G2 to be incident as the second measurement light on the second imager 14. The predetermined light G2 is light causing error information. However, as described above, it is possible to reduce the predetermined light G2. Thus, it is possible to perform distance measurement with high accuracy.

As described above, the wavelength band of a part of the wavelength band of the predetermined light G2 is the same as the wavelength band of the second irradiation light G2′, and the remaining wavelength band is different from the wavelength band of the second irradiation light G2′.

If the spectral characteristics of the bandpass filter 16 are changed to transmit only light in the same wavelength band as the wavelength band of the second irradiation light G2′, light in the remaining wavelength band is also reflected by the bandpass filter 16.

In the optical device 20 illustrated in FIG. 2, the bandpass filter 16 may be disposed between the second optical system 23 and the second imager 14.

When the first light source and the second light source are simultaneously turned on, the subject is irradiated with the first irradiation light and the second irradiation light simultaneously. In this case, the use of a dichroic mirror is effective in reducing the predetermined light in the second measurement light. However, it is not possible to completely eliminate the predetermined light.

Furthermore, the use of the bandpass filter 16 is effective in eliminating the remaining light in the second measurement light. However, it is not possible to completely eliminate the predetermined light.

Then, as described above, the peak wavelength of the second irradiation light is positioned between adjacent peak wavelengths of the first irradiation light. The light intensity of the first irradiation light is low between adjacent peak wavelengths of the first irradiation light. Positioning the peak wavelength of the second irradiation light in a wavelength band in which the light intensity of the first irradiation light is low is effective for further reducing the predetermined light in the second measurement light.

Half mirrors may be used for the dichroic mirror 35 and the optical filter 12. When the first light source and the second light source are simultaneously turned on, the subject is irradiated with the first irradiation light and the second irradiation light simultaneously. When half mirrors are used, the first irradiation light R, the first irradiation light G1, the predetermined light G2, the first irradiation light B, and the second irradiation light G2′ travel toward the second imager 14.

Also in this case, by disposing the bandpass filter 16, it is possible to reflect the first irradiation light R, the first irradiation light G1, and the first irradiation light B by the bandpass filter 16. As a result, it is possible to allow only the second irradiation light G2′ and the predetermined light G2 to be incident as the second measurement light on the second imager 14.

### (Optical Device 6 of the Present Embodiment)

In the optical device of the present embodiment, it is preferable that turning-on of the first light source and turning-on of the second light source be alternately performed.

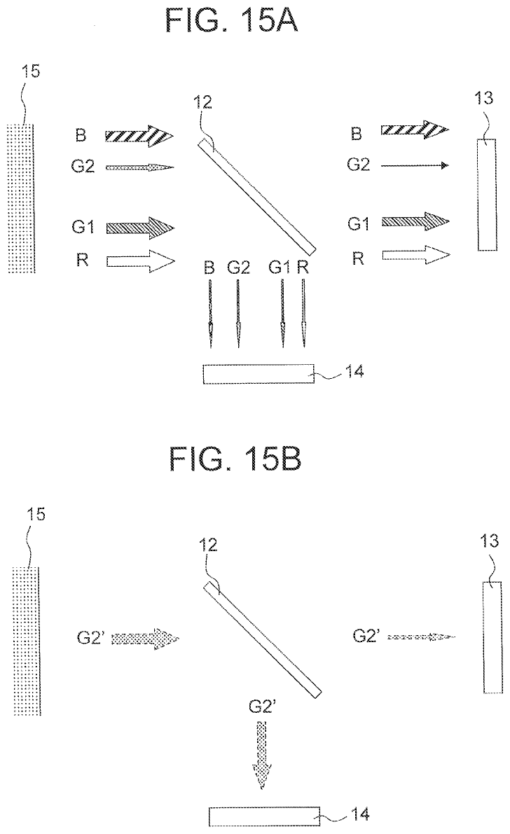

FIG. 15A and FIG. 15B are diagrams illustrating measurement light. FIG. 15A is a diagram illustrating measurement light in a first state. FIG. 15B is a diagram illustrating measurement light in a second state. The same component as that in FIG. 14 is denoted by the same numeral and a description thereof is omitted.

In this optical device, it is possible to alternately perform turning-on of the first light source and turning-on of the second light source. The alternate turning-on brings about the first state and the second state.

In the first state, the first light source turns on, and the second light source turns off. Hence, as illustrated in FIG. 15A, the first irradiation light (the first irradiation light R, the first irradiation light G1, the predetermined light G2, and the first irradiation light B) is incident on the first imager 13 and the second imager 14.

An optical image by the first irradiation light is formed on the first imager 13. Acquisition of an optical image is performed in the first imager 13. As a result, image information of the subject is output from the first imager 13.

An optical image by the first irradiation light is formed on the second imager 14. The first irradiation light is light causing error information. While an optical image by the light causing error information is formed on the second imager 14, acquisition of an optical image is not performed in the second imager 14. As a result, neither distance information nor error information is output from the second imager 14.

In the second state, the first light source turns off, and the second light source turns on. Hence, as illustrated in FIG. 15B, the second irradiation light (the second irradiation light G2′) is incident on the first imager 13 and the second imager 14.

An optical image by the second irradiation light is formed on the first imager 13. Acquisition of an optical image is not performed in the first imager 13. As a result, image information of the subject is not output from the first imager 13.

An optical image by the second irradiation light is formed on the second imager 14. Acquisition of an optical image is performed in the second imager 14. As a result, distance information is output from the second imager 14.

In the second state, since the first irradiation light does not exist, an optical image by the first irradiation light is not formed on the second imager 14. That is, an optical image by the light causing error information is not formed on the second imager 14. In this case, even when acquisition of an optical image is performed in the second imager 14, distance information output from the second imager 14 does not include error information. Thus, it is possible to perform distance measurement with high accuracy.

In the optical device, the first irradiation light and the second irradiation light are emanated from the light collecting unit. The first irradiation light and the second irradiation light are incident on the incident end face of the light guide member. The light guide member will be described below.

### (Optical Device 7 of the Present Embodiment)

In the optical device of the present embodiment, it is preferable that the insertion section have one incident end face, the one incident end face include a first incident region and a second incident region, the first irradiation light be incident on the first incident region, and the second irradiation light be incident on the second incident region.

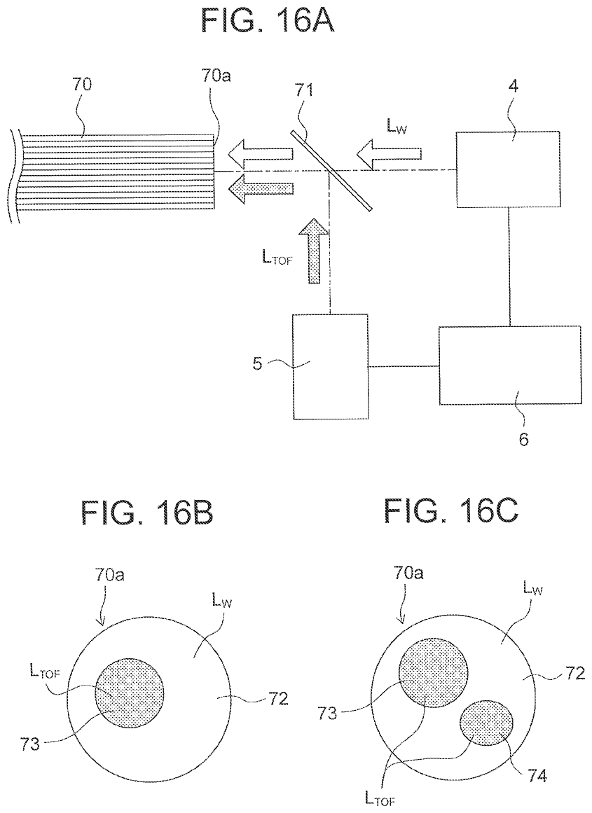

FIG. 16A, FIG. 16B, and FIG. 16C are diagrams illustrating the incident end face and the incident region. FIG. 16A is a diagram illustrating the incident end face. FIG. 16B is a diagram illustrating a first example of the incident region. FIG. 16C is a diagram illustrating a second example of the incident region. The same component as that in FIG. 1A is denoted by the same numeral and a description thereof is omitted.

In this optical device, a light guide member 70 and a parallel flat plate 71 are disposed on the light source unit side. As illustrated in FIG. 16A, the light guide member 70 has an incident end face 70a. The parallel flat plate 71 is disposed on the incident end face 70a side. First irradiation light L_{W} and second irradiation light L_{TOF} are incident on the incident end face 70a.

As illustrated in FIG. 16B, the incident end face 70a includes a first incident region 72 and a second incident region 73. The first irradiation light L_{W} is incident on the first incident region 72. The second irradiation light L_{TOF} is incident on the second incident region 73.

As illustrated in FIG. 16C, the incident end face 70a includes the first incident region 72, the second incident region 73, and a second incident region 74. The first irradiation light L_{W} is incident on the first incident region 72. The second irradiation light L_{TOF} is incident on the second incident region 73 and the second incident region 74.

In the incident end face 70a illustrated in FIG. 16B, the number of first incident regions and the number of second incident regions are both one. In the incident end face 70a illustrated in FIG. 16C, the number of first incident regions is one and the number of second incident regions is two.

It is possible to use a dichroic mirror or a half mirror as the parallel flat plate 71. When the parallel flat plate 71 is a dichroic mirror, the first irradiation light L_{W} is not incident on the second region. Only the second irradiation light L_{TOF} is incident on the second region. When the parallel flat plate 71 is a half mirror, not only the second irradiation light L_{TOF} but also the first irradiation light L_{W} is incident on the second region.

When the parallel flat plate 71 is a half mirror, a light-shielding member may be disposed between the first light source 4 and the parallel flat plate 71. In the light-shielding member, light of a portion corresponding to the second region is blocked. By doing so, the first irradiation light L_{W} is not incident on the second region. Thus, it is possible to allow only the second irradiation light L_{TOF} to be incident on the second region.

In this optical device, the same light guide member is commonly used for guiding the first irradiation light and guiding the second irradiation light. The configuration of the light guide member is as described in the overall shape 1 of the light guide member (described later). Since the insertion section and the light guide member continuous thereto can be commonly used, this configuration is effective for reducing the diameter.

In this optical device, even when there are a plurality of exit end faces as in an optical device 11 described later, light is guided to a predetermined one of the exit end faces. Then, the second irradiation light is incident on the second region of the incident end face and is emanated from one exit end face, whereby not only the diameter is reduced but also distance measurement can be performed accurately.

### (Optical Device 8 of the Present Embodiment)

In the optical device of the present embodiment, it is preferable that the insertion section have a plurality of incident end faces, the incident end faces be spatially separated, and the incident end face on which the first irradiation light is incident be different from the incident end face on which second irradiation light is incident.

In this optical device, it is possible to use the light source unit 37 illustrated in FIG. 3B. The light source unit 37 is a parallel incident-type light source unit. In the light source unit 37, two light guide members are disposed on the light source unit side. The light source unit 37 includes the light guide member 38 and the light guide member 39. The light guide member 38 and the light guide member 39 are disposed in the insertion section.

The light guide member 38 has an incident end face 38a. The light guide member 39 has an incident end face 39a. In this way, in the optical device, the insertion section has two incident end faces.

In the optical device, the incident end face 38a and the incident end face 39a are spatially separated. The first irradiation light L_{W} is incident on the incident end face 38a. The second irradiation light L_{TOF} is incident on the incident end face 39a. The incident end face 38a is an incident end face on which the first irradiation light L_{W} is incident. The incident end face 39a is an incident end face on which the second irradiation light L_{TOF} is incident.

In this optical device, since two incident end faces are spatially separated, it is possible to allow the first irradiation light and the second irradiation light to be incident on the light guide members without using a coaxial incident-type light source unit (see FIG. 3A).

Furthermore, it is possible to provide an exit end face corresponding one-to-one to the incident end face. In this case, it is possible to reliably emit only the second irradiation light L_{TOF} from the light guide member.

The wording “a plurality of incident end faces are spatially separated” means that, for example, when a plurality of incident end faces include a first incident end face and a second incident end face, a light guide member having the first incident end face and a light guide member having the second incident end face function independently. A space may be formed between two light guide members or two light guide members may be in contact with each other.

### (Optical Device 8: Thirteenth Example)

In the optical device of the present embodiment, it is preferable that the second light source be disposed in the body unit.

In this optical device, it is possible to use the light source unit 37 illustrated in FIG. 3B. The light source unit 37 is a parallel incident-type light source unit. In the light source unit 37, the light guide member 38 has the incident end face 38a. The light guide member 39 has the incident end face 39a. As just described, in the light source unit 37, the optical device has the incident end face 38a and the incident end face 39a. The incident end face 38a is a first incident end face. The incident end face 39a is a second incident end face.

The incident end face 38a and the incident end face 39a are spatially separated. The first irradiation light L_{W} is incident on the incident end face 38a. The second irradiation light L_{TOF} is incident on the incident end face 39a.

In the light source unit 37, the light guide member that allows the second irradiation light L_{TOF} to be incident is different from the light guide member that allows the first irradiation light L_{W} to be incident. Hence, when the light source unit 37 is used in the optical device, it is possible to dispose only the second light source 32 in the interior of the body unit 3.

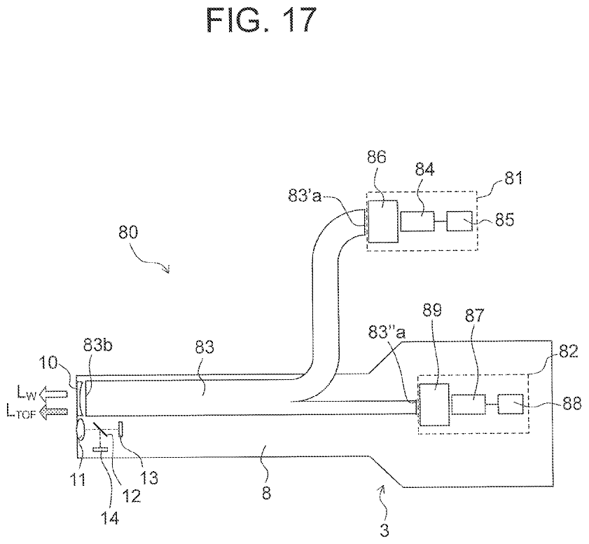

FIG. 17 is a diagram illustrating an optical device. The same component as that in FIG. 1A is denoted by the same numeral and a description thereof is omitted.

An optical device 80 includes a first light source unit 81, a second light source unit 82, and the body unit 3. In the optical device 80, the first light source unit 81 is disposed at a place away from the body unit 3. The second light source unit 82 is disposed in the interior of the body unit 3.

The first light source unit 81 includes a first light source 84, a first light source control unit 85, and a first light collecting unit 86. The second light source unit 82 includes a second light source 87, a second light source control unit 88, and a second light collecting unit 89.

In the optical device 80, the body unit 3 includes a light guide member 83. The light guide member 83 is split into two light guide members on the light source unit side. Thus, the light guide member 83 has a first incident end face 83′a, a second incident end face 83″a, and an exit end face 83b.

The first incident end face 83′a faces the first light collecting unit 86. The second incident end face 83″a faces the second light collecting unit 89. The exit end face 83b faces the lens 10.

As described above, the longer the entire length of the light guide member is, the greater the error d is. In the optical device 80, the second light source unit 82 is disposed in the interior of the body unit 3. Hence, the length from the second incident end face 83″a to the exit end face 83b is shorter than the length from the first incident end face 83′a to the exit end face 83b. Thus, in the optical device of the present embodiment, it is possible to diminish the change in pulse shape. As a result, it is possible to reduce error information.

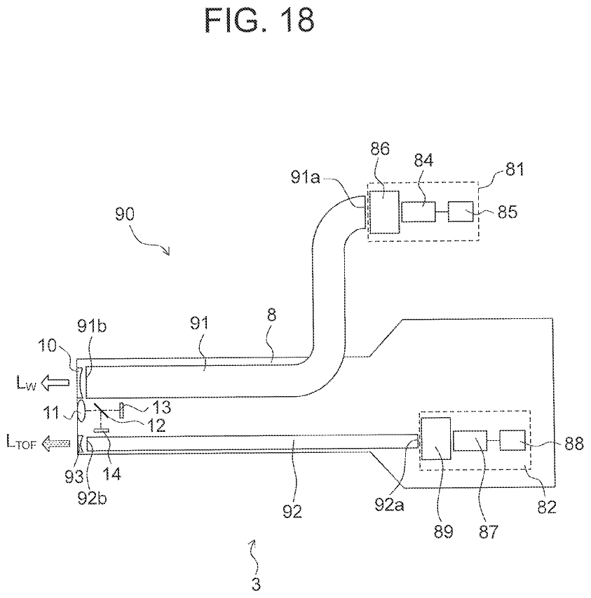

FIG. 18 is a diagram illustrating an optical device. The same component as that in FIG. 1A and FIG. 17 is denoted by the same numeral and a description thereof is omitted.

An optical device 90 includes the first light source unit 81, the second light source unit 82, and the body unit 3. In the optical device 90, the first light source unit 81 is disposed at a place away from the body unit 3. The second light source unit 82 is disposed in the interior of the body unit 3.

In the optical device 90, the body unit 3 includes a light guide member 91 and a light guide member 92. The light guide member 91 has an incident end face 91a and an exit end face 91b. The light guide member 92 has an incident end face 92a and an exit end face 92b.

The incident end face 91a faces the first light collecting unit 86. The incident end face 92a faces the second light collecting unit 89. The exit end face 91b faces the lens 10. The exit end face 92b faces a lens 93.

As described above, the longer the entire length of the light guide member is, the greater the error d is. In the optical device 90, the second light source unit 82 is disposed in the interior of the body unit 3. Hence, the length from the incident end face 92a to the exit end face 92b is shorter than the length from the incident end face 91a to the exit end face 91b. Thus, in the optical device of the present embodiment, it is possible to diminish the change in pulse shape. As a result, it is possible to reduce error information.

### (Optical Device 8: Fourteenth Example)

In the optical device of the present embodiment, it is preferable that the incident angle of the second irradiation light at an incident end face on which the second irradiation light is incident be 9.9° or less.

It is possible to use the optical device 80 (FIG. 17) or the optical device 90 (FIG. 18) as the optical device. In the optical device 80 or the optical device 90, it is possible to reduce the length of the light guide member that propagates the second irradiation light L_{TOF}.

It is possible to use this optical device in a flexible endoscope. In this case, the optical device 80 or the optical device 90 is used in a flexible endoscope. In the optical device 80 and the optical device 90, the light source unit 60 (see FIG. 7) is used.

As illustrated in FIG. 7, the second irradiation light L_{TOF} is incident in a collected state on the incident end face 67a. Thus, the second irradiation light L_{TOF} is incident on the incident end face 67a at various angles from 0° to θ2.

Even with this optical device, when the size of a lesion area can be measured within an error of about 10%, it is possible to use the measurement result for a definitive diagnosis of the lesion area. As described above, in order to keep an error within 10% at the distance of 5 cm, it is necessary to set d to 10 mm or less when the error is 5 mm.

As described above, the optical device 80 or the optical device 90 is used in a flexible endoscope. In such a flexible endoscope, it is possible to install the second light source unit 82 in a manipulator of the endoscope. In this case, the value of L is 1000 mm. When n=1.5 and d=10 mm, θ≈9.9°.

Thus, when the optical device 80 is used in a flexible endoscope, the incident angle of the second irradiation light L_{TOF} on the incident end face 83″a may be set to 9.9° or less. By doing so, it is possible to diminish the change in pulse shape. As a result, it is possible to reduce error information.

The manipulator of the endoscope is installed at a part of the body unit 3. The manipulator is used by the user to grip the endoscope and to manipulate the insertion section. It is possible to keep a space for accommodating the second light source unit 82 in the interior of the manipulator or in the periphery of the manipulator. Thus, by disposing the second light source unit 82 in the interior of the body unit 3, it is possible to decrease the value of L, compared with when the second light source unit 82 is disposed on the incident end face 83′a side.

### (Optical Device 8: Fifteenth Example)

In the optical device of the present embodiment, it is preferable that the incident angle of the second irradiation light at an incident end face on which the second irradiation light is incident be 4.4° or less.

As described above, in a flexible endoscope, a surface of the upper digestive tract is sometimes observed at a distance of about 1 cm. In this case, in order to keep an error within 10% or less, it is necessary to set d to 0.2 mm or less.

As described above, the optical device 80 or the optical device 90 is used in a flexible endoscope. In such a flexible endoscope, the value of L is 1000 mm. When n=1.5 and d=0.2 mm, θ≈4.4°.

Thus, when the optical device 90 is used in a flexible endoscope, the incident angle of the second irradiation light L_{TOF} on the incident end face 92a is set to 4.4° or less. By doing so, it is possible to diminish the change in pulse shape. As a result, it is possible to reduce error information.

### (Optical Device 8: Sixteenth Example)

In the optical device of the present embodiment, it is preferable that the area of the second incident end face be smaller than the area of the first incident end face.

It is possible to use the light source unit 37 (see FIG. 3B) in the optical device. As described above, the light source unit 37 has the incident end face 38a and the incident end face 39a. The incident end face 38a is a first incident end face. The incident end face 39a is a second incident end face. The first irradiation light L_{W} is incident on the incident end face 38a. The second irradiation light L_{TOF} is incident on the incident end face 39a.

When the optical device has two incident end faces, as illustrated in FIG. 17 and FIG. 18, it is possible to dispose one of the incident end faces in the interior of the body unit.

As described above, the second irradiation light L_{TOF} is incident on the incident end face 39a, that is, the second incident end face. Furthermore, in order to reduce error information, the entire length of the light guide member that propagates the second irradiation light L_{TOF} should be short. Thus, the second incident end face should be disposed in the interior of the body unit.

However, it is desirable that the body unit be compact. In this optical device, the area of the second incident end face is smaller than the area of the first incident end face. Hence, it is possible to reduce error information without increasing the size of the body unit.

### (Optical Device 9)

In the optical device of the present embodiment, it is preferable that the insertion section have one exit end face, the exit end face include a first exit region and a second exit region, the first irradiation light be emanated from the first exit region, and the second irradiation light be emanated from the second exit region.

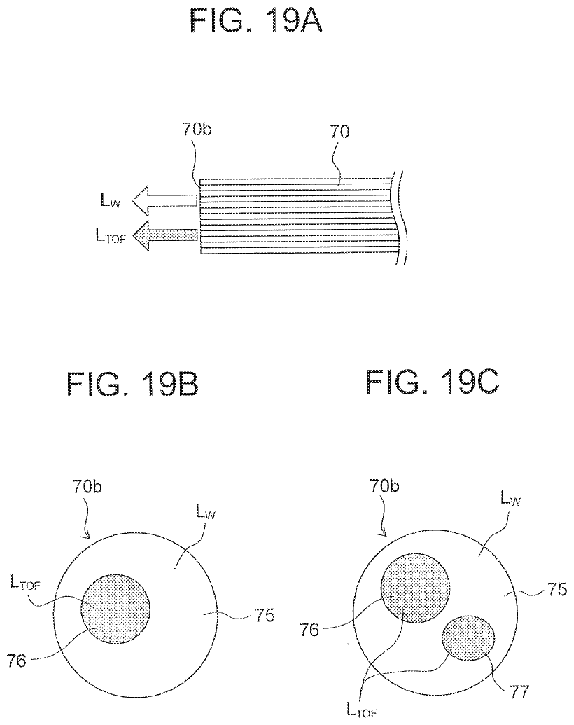

FIG. 19A, FIG. 19B, and FIG. 19C are diagrams illustrating an exit end face and an exit region. FIG. 19A is a diagram illustrating the exit end face. FIG. 19B is a diagram illustrating a first example of the exit region. FIG. 19C is a diagram illustrating a second example of the exit region. A description will be given using the light guide member 70 illustrated in FIG. 16A.

In this optical device, one light guide member is disposed on the subject side. As illustrated in FIG. 19A, the light guide member 70 has an exit end face 70b. The first irradiation light L_{W} and the second irradiation light L_{TOF} are emanated from the exit end face 70b.

As illustrated in FIG. 19B, the exit end face 70b includes a first exit region 75 and a second exit region 76. The first irradiation light L_{W} is emanated from the first exit region 75. The second irradiation light L_{TOF} is emanated from the second exit region 76.

As illustrated in FIG. 19C, the exit end face 70b has the first exit region 75, the second exit region 76, and a second exit region 77. The first irradiation light L_{W} is emanated from the first exit region 75. The second irradiation light L_{TOF} is emanated from the second exit region 76 and the second exit region 77.

In the exit end face 70b illustrated in FIG. 19B, the number of first exit regions and the number of second exit regions are both one. In the exit end face 70b illustrated in FIG. 19C, the number of first exit regions is one, and the number of second exit regions is two.

The number of incident end faces is not limited to one. A light guide member having one exit end face and a plurality of incident end faces may be used. For example, it is possible to use the light guide member 83 (see FIG. 17) instead of the light guide member 70.

### (Optical Device 10)

In the optical device of the present embodiment, it is preferable that the insertion section have a plurality of exit end faces, the exit end faces be spatially separated, and the exit end face from which the first irradiation light is emanated be different from the exit end face from which the second irradiation light is emanated.

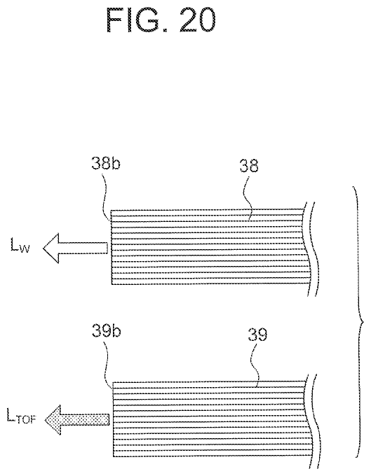

FIG. 20 is a diagram illustrating the exit end face. A description will be given using the light guide member 38 and the light guide member 39 illustrated in FIG. 3B.

In this optical device, two light guide members are disposed on the subject side. As illustrated in FIG. 20, the optical device includes the light guide member 38 and the light guide member 39. The light guide member 38 and the light guide member 39 are disposed in the insertion section.

The light guide member 38 has an exit end face 38b. The light guide member 39 has an exit end face 39b. As just described, in this optical device, the insertion section has two exit end faces.

In this optical device, the exit end face 38b and the exit end face 39b are spatially separated. The first irradiation light L_{W} is emanated from the exit end face 38b. The second irradiation light L_{TOF} is emanated from the exit end face 39b. The exit end face 38b is an exit end face from which the first irradiation light L_{W} is emanated. The exit end face 39b is an exit end face from which the second irradiation light L_{TOF} is emanated.

The wording “a plurality of exit end faces are spatially separated” means that, for example, when a plurality of exit end faces include a first exit end face and a second exit end face, a light guide member having the first exit end face and a light guide member having the second exit end face function independently. A space may be formed between two light guide members or two light guide members may be in contact with each other.

The number of incident end faces is not limited to one. A light guide member having two exit end faces and a plurality of incident end faces may be used.

### (Overall Shape of Light Guide Member)

As described above, it is possible that the number of incident end faces and the number of exit end faces each is one or more. Hence, it is possible to form the overall shape of the light guide member in various shapes.

### (Overall Shape 1 of Light Guide Member)

In the light guide member in the optical device of the present embodiment, it is preferable that the first irradiation light and the second irradiation light be incident on one light guide member.

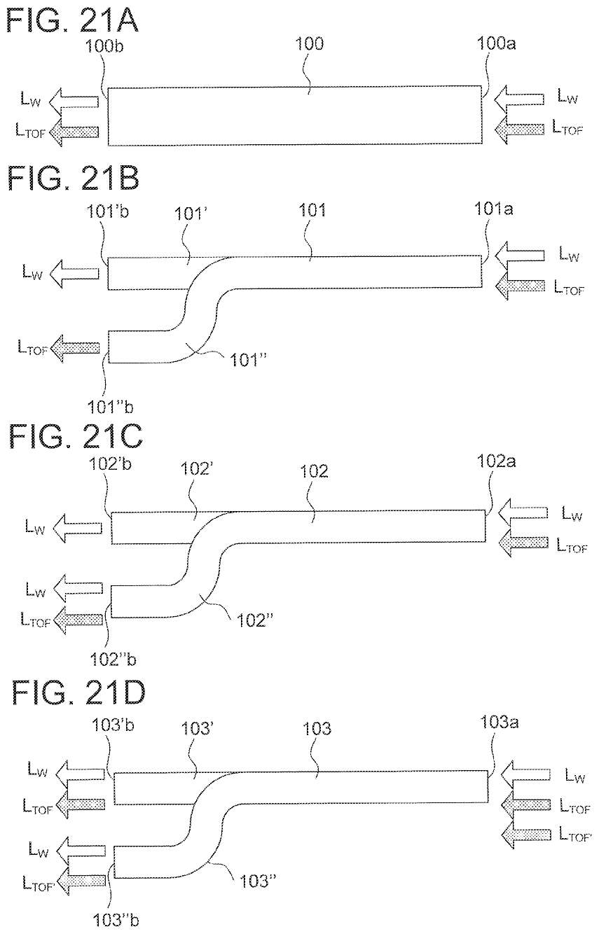

The light guide member will be described. FIG. 21A, FIG. 21B, FIG. 21C, and FIG. 21D are diagrams illustrating a light guide member. FIG. 21A is a diagram illustrating a first example of the light guide member. FIG. 21B is a diagram illustrating a second example of the light guide member. FIG. 21C is a diagram illustrating a third example of the light guide member. FIG. 21D is a diagram illustrating a fourth example of the light guide member.

As illustrated in FIG. 21A, a light guide member 100 has an incident end face 100a and an exit end face 100b. In the light guide member 100, the number of incident end faces and the number of exit end faces are both one. The first irradiation light L_{W} and the second irradiation light L_{TOF} are incident together on the incident end face 100a. The first irradiation light L_{W} and the second irradiation light L_{TOF} are emanated together from the exit end face 100b.

By using the light guide member 100, it is possible to make the insertion section thinner, compared with a light guide member 104 described later.

As illustrated in FIG. 21B, a light guide member 101 has an incident end face 101a, an exit end face 101′b, and an exit end face 101″b. The light guide member 101 is split into a light guide member 101′ and a light guide member 101″ on the subject side. The light guide member 101′ has the exit end face 101′b. The light guide member 101″ has the exit end face 101″b.

In the light guide member 101, the number of incident end faces is one, and the number of exit end faces is two. The first irradiation light L_{W} and the second irradiation light L_{TOF} are incident together on the incident end face 101a. The first irradiation light L_{W} is emanated from the exit end face 101′b. The second irradiation light L_{TOF} is emanated from the exit end face 101″b.

As illustrated in FIG. 21C, a light guide member 102 has an incident end face 102a, an exit end face 102′b, and an exit end face 102″b. The light guide member 102 is split into a light guide member 102′ and a light guide member 102″ on the subject side. The light guide member 102′ has the exit end face 102′b. The light guide member 102″ has the exit end face 102″b.

In the light guide member 102, the number of incident end faces is one, and the number of exit end faces is two. The first irradiation light L_{W} and the second irradiation light L_{TOF} are incident together on the incident end face 102a. The first irradiation light L_{W} is emanated from the exit end face 102′b. The first irradiation light L_{W} and the second irradiation light L_{TOF} are emanated from the exit end face 102″b.

It is possible to use the optical device including the light guide member 102 in an endoscope. In an endoscope, the first irradiation light L_{W} is often emanated from a plurality of exit end faces in order to obtain an image free from shadows or an image free from unevenness in brightness. In the light guide member 102, the first irradiation light L_{W} is emanated from two exit end faces. Hence, it is possible to obtain an image free from shadows or an image free from unevenness in brightness.

In the light guide member 102, the second irradiation light L_{TOF} is emanated only from the exit end face 102″b. In FIG. 16B, by allowing the second irradiation light L_{TOF} to be incident on the second incident region 73, it is possible to select the exit end face 102″b as an exit end face for the second irradiation light L_{TOF}.

As illustrated in FIG. 21D, a light guide member 103 has an incident end face 103a, an exit end face 103′b, and an exit end face 103″b. The light guide member 103 is split into a light guide member 103′ and a light guide member 103″ on the subject side. The light guide member 103′ has the exit end face 103′b. The light guide member 103″ has the exit end face 103″b.

In the light guide member 103, the number of incident end faces is one, and the number of exit end faces is two. The first irradiation light L_{W}, the second irradiation light L_{TOF}, and second irradiation light L_{TOF}′ are incident together on the incident end face 103a. The first irradiation light L_{W} and the second irradiation light L_{TOF} are emanated from the exit end face 103′b. The first irradiation light L_{W} and the second irradiation light L_{TOF}′ are emanated from the exit end face 103″b. The wavelength band of the second irradiation light L_{TOF}′ is different from the wavelength band of the second irradiation light L_{TOF}.

In the light guide member 103, the first irradiation light L_{W} is emanated from two exit end faces, in the same manner as in the light guide member 102. Hence, by using the optical device including the light guide member 103 in an endoscope, it is possible to obtain an image free from shadows or an image free from unevenness in brightness.

In the light guide member 103, the second irradiation light L_{TOF} and the second irradiation light L_{TOF}′ can be emanated. Hence, for example, even when distance measurement is difficult with the second irradiation light L_{TOF}, it is possible to perform distance measurement with the second irradiation light L_{TOF}′.

The wavelength band of the second irradiation light L_{TOF} and the wavelength band of the second irradiation light L_{TOF}′ may be the same or may be different. Emanation of the second irradiation light L_{TOF} and emanation of the second irradiation light L_{TOF}′ are not performed simultaneously.

In the light guide member 103, the second irradiation light L_{TOF} is emanated from the exit end face 103′b, and the second irradiation light L_{TOF}′ is emanated from the exit end face 103″b. In FIG. 16C, by allowing the second irradiation light L_{TOF} to be incident on the second incident region 73, it is possible to select the exit end face 103′b as an exit end face for the second irradiation light L_{TOF}. When the second irradiation light L_{TOF}′ is allowed to be incident on the second incident region 74, it is possible to select the exit end face 103″b as an exit end face for the second irradiation light L_{TOF}′.

### (Overall Shape 2 of Light Guide Member)

In the light guide member in the optical device of the present embodiment, it is preferable that the first irradiation light and the second irradiation light be incident on different incident end faces.

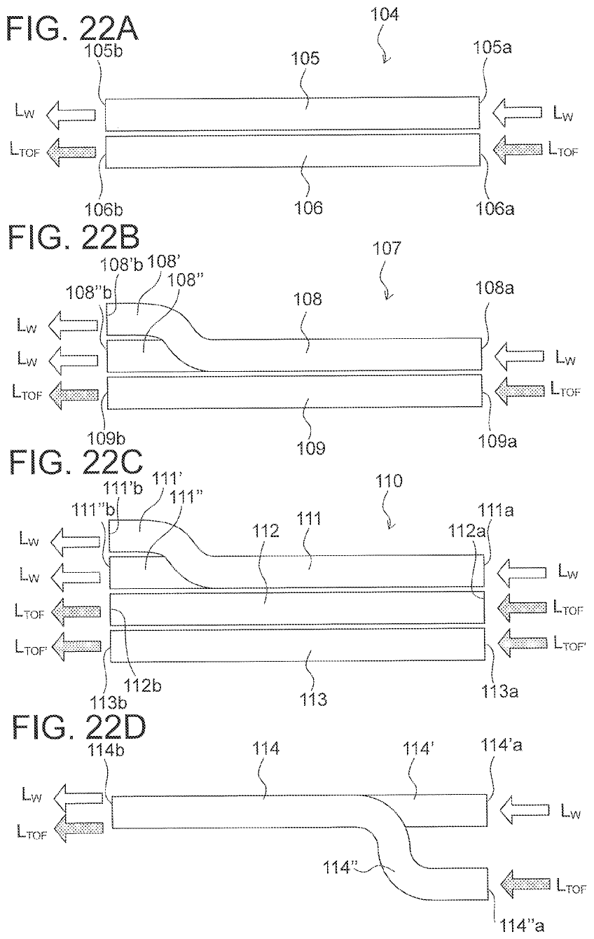

The light guide member will be described. FIG. 22A, FIG. 22B, FIG. 22C, and FIG. 22D are diagrams illustrating the light guide member. FIG. 22A is a diagram illustrating a fifth example of the light guide member. FIG. 22B is a diagram illustrating a sixth example of the light guide member. FIG. 22C is a diagram illustrating a seventh example of the light guide member. FIG. 22D is a diagram illustrating an eighth example of the light guide member.

As illustrated in FIG. 22A, a light guide member 104 has an incident end face 105a, an incident end face 106a, an exit end face 105b, and an exit end face 106b. The light guide member 104 is split into a light guide member 105 and a light guide member 106. The light guide member 105 has an incident end face 105a and an exit end face 105b. The light guide member 106 has an incident end face 106a and an exit end face 106b.

In the light guide member 104, the number of incident end faces is two, and the number of exit end faces is two. The first irradiation light L_{W} is incident on the incident end face 105a. The second irradiation light L_{TOF} is incident on the incident end face 106a. The first irradiation light L_{W} is emanated from the exit end face 105b. The second irradiation light L_{TOF} is emanated from the exit end face 106b.

As illustrated in FIG. 22B, a light guide member 107 has an incident end face 108a, an incident end face 109a, an exit end face 108′b, an exit end face 108″b, and an exit end face 109b.

The light guide member 107 is split into a light guide member 108 and a light guide member 109. The light guide member 108 is split into a light guide member 108′ and a light guide member 108″ on the subject side. The light guide member 108 has the incident end face 108a, the exit end face 108′b, and the exit end face 108″b. The light guide member 109 has the incident end face 109a and the exit end face 109b.

In the light guide member 107, the number of incident end faces is two, and the number of exit end faces is three. The first irradiation light L_{W} is incident on the incident end face 108a. The second irradiation light L_{TOF} is incident on the incident end face 109a. The first irradiation light L_{W} is emanated from the exit end face 108′b and the exit end face 108″b. The second irradiation light L_{TOF} is emanated from the exit end face 109b.

In the light guide member 107, the first irradiation light L_{W} is emanated from two exit end faces, in the same manner as in the light guide member 102. Hence, by using the optical device including the light guide member 107 in an endoscope, it is possible to obtain an image free from shadows or an image free from unevenness in brightness.

As illustrated in FIG. 22C, a light guide member 110 has an incident end face 111a, an incident end face 112a, an incident end face 113a, an exit end face 111′b, an exit end face 111″b, an exit end face 112b, and an exit end face 113b.

The light guide member 110 is split into a light guide member 111, a light guide member 112, and a light guide member 113. The light guide member 111 is split into a light guide member 111′ and a light guide member 111″ on the subject side. The light guide member 111 has the incident end face 111a, the exit end face 111′b, and the exit end face 111″b.

The light guide member 112 has the incident end face 112a and the exit end face 112b. The light guide member 113 has the incident end face 113a and the exit end face 113b.

In the light guide member 110, the number of incident end faces is three, and the number of exit end faces is four. The first irradiation light L_{W} is incident on the incident end face 111a. The second irradiation light L_{TOF} is incident on the incident end face 112a. The second irradiation light L_{TOF}′ is incident on the incident end face 113a.

The first irradiation light L_{W} is emanated from the exit end face 111′b and the exit end face 111″b. The second irradiation light L_{TOF} is emanated from the exit end face 112b. The second irradiation light L_{TOF}′ is emanated from the exit end face 113b.

In the light guide member 110, the first irradiation light L_{W} is emanated from two exit end faces, in the same manner as in the light guide member 102. Hence, by using the optical device including the light guide member 110 in an endoscope, it is possible to obtain an image free from shadows or an image free from unevenness in brightness.

In the light guide member 110, the second irradiation light L_{TOF} and the second irradiation light L_{TOF}′ can be emanated, in the same manner as in the light guide member 103. Hence, for example, even when distance measurement is difficult with the second irradiation light L_{TOF}, it is possible to perform distance measurement with the second irradiation light L_{TOF}′.

The wavelength band of the second irradiation light L_{TOF} and the wavelength band of the second irradiation light L_{TOF}′ may be the same or may be different. Emanation the second irradiation light L_{TOF} and emanation of the second irradiation light L_{TOF}′ are not performed simultaneously.

As illustrated in FIG. 22D, a light guide member 114 has an incident end face 114′a, an incident end face 114″a, and an exit end face 114b. The light guide member 114 is split into a light guide member 114′ and a light guide member 114″ on the incident end side. The light guide member 114′ has the incident end face 114′a. The light guide member 114″ has the incident end face 114″a.

In the light guide member 114, the number of incident end faces is two, and the number of exit end faces is one. The first irradiation light L_{W} is incident on the incident end face 114′a. The second irradiation light L_{TOF} is incident on the incident end face 114″a. The first irradiation light and the second irradiation light L_{TOF} are emanated together from the exit end face 114b.

### (Optical Device 11)

In the optical device of the present embodiment, it is preferable that the insertion section have a plurality of exit end faces, and the second irradiation light be emanated only from predetermined one exit end face.

In the optical device, it is possible that the insertion section has a plurality of exit end faces. In this case, as illustrated in FIG. 21C or FIG. 22B, the first irradiation light L_{W} is emanated from a plurality of exit end faces, and the second irradiation light L_{TOF} is emanated only from a single exit end face. That is, the second irradiation light L_{TOF} is prevented from being emanated simultaneously from two or more exit end faces.

The first irradiation light L_{W} is used for image acquisition. As described above, in an endoscope, the first irradiation light L_{W} is often emanated from a plurality of exit end faces in order to obtain an image free from shadows or an image free from unevenness in brightness. On the other hand, the second irradiation light L_{TOF} is used for distance measurement.

When the second irradiation light L_{TOF} is emanated from a plurality of exit end faces, the individual rays of the second irradiation light L_{TOF} reach the second imager through different paths. In this case, the individual rays of the second irradiation light L_{TOF} have different time delays. Hence, when the individual rays of the second irradiation lights L_{TOF} are combined, the shape of the combined pulsed light differs from the shape of the pulsed light emitted from the second light source. Hence, it is not possible to measure the precise distance with the combined pulsed light.

Since even the pulsed light emanated from one exit end face uses the return light from the subject of the light obliquely emanated from the exit end face, the distance between each point of the subject and the end of the optical device does not have a simple proportional relation to a time delay of the pulsed light. However, if the pulsed light is from one exit end face, it is not a combination of rays of pulsed light having two different time delays. In this case, it is possible to measure a precise time delay. Hence, it is possible to determine the distance in accordance with a table defined for each pixel.

In the light guide member 102 illustrated in FIG. 21C, the light intensity distribution of the second irradiation light L_{TOF} sometimes has a long tail to the periphery, like a Gaussian distribution. In this case, the second irradiation light L_{TOF} may be unable to be entirely incident on the incident region 73 illustrated in FIG. 16B, and light on the periphery of the light intensity distribution may be incident on the outside of the incident region 73.

If so, for example, when the light guide member 102 illustrated in FIG. 21C is used as the light guide member of the optical device, the second irradiation light L_{TOF} may be emanated not only from the exit end face 102″b but also from the exit end face 102′b.

As described above, if the second irradiation light L_{TOF} is emanated from a plurality of exit end faces, it is not possible to measure a precise distance. However, as long as the proportion of the second irradiation light L_{TOF} from the exit end face 102′b is approximately 10% or less, distance measurement can be performed accurately.

### (Optical Device 12: Seventeenth Example)

In the optical device of the present embodiment, it is preferable that the insertion section have a plurality of exit end faces, the second irradiation light be emanated from two or more exit end faces, and the second irradiation light be emanated from only one exit end face at the same time.

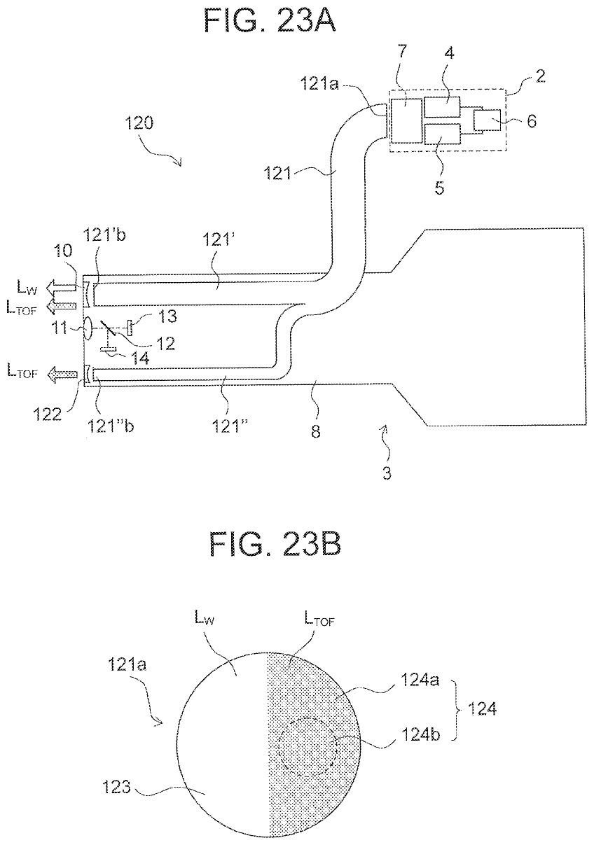

FIG. 23A and FIG. 23B are diagrams illustrating an optical device of the present embodiment and an incident region. FIG. 23A is a diagram illustrating the optical device. FIG. 23B is a diagram illustrating the incident region. The same component as that in FIG. 1A is denoted by the same numeral and a description thereof is omitted.

An optical device 120 includes the light source unit 2 and the body unit 3. In the optical device 120, the light source unit 2 is disposed at a place away from the body unit 3.

In the optical device 120, the body unit 3 includes a light guide member 121. The light guide member 121 has an incident end face 121a, an exit end face 121′b, and an exit end face 121″b. The optical device 120 has one incident end face and two exit end faces.

The light guide member 121 is split into a light guide member 121′ and a light guide member 121″ on the subject side. The light guide member 121′ and the light guide member 121″ are disposed in the insertion section 8. The light guide member 121′ has the exit end face 121′b. The light guide member 121″ has the exit end face 121″b. In the optical device 120, the insertion section 8 has two exit end faces.

The incident end face 121a faces the light collecting unit 7. The exit end face 121′b faces the lens 10. The exit end face 121″b faces a lens 122.

The first irradiation light L_{W} and the second irradiation light L_{TOF} are incident on the incident end face 121a. As illustrated in FIG. 23B, the incident end face 121a is split into a first incident region 123 on which the first irradiation light L_{W} is incident and a second incident region 124 on which the second irradiation light L_{TOF} is incident. The second incident region 124 is split into an incident region 124a and an incident region 124b.

The exit region corresponding to the first incident region 123 is positioned at the exit end face 121′b. The exit region corresponding to the second incident region 124 is positioned at the exit end face 121′b and the exit end face 121″b. For example, the exit region corresponding to the incident region 124a is positioned at the exit end face 121′b. The exit region corresponding to the incident region 124b is positioned at the exit end face 121″b.

Thus, the first irradiation light L_{W} and the second irradiation light L_{TOF} are emanated from the exit end face 121′b. Only the second irradiation light L_{TOF} is emanated from the exit end face 121″b.

In the optical device 120, the second irradiation light L_{TOF} can be emanated from two exit end faces. As a result, in the optical device 120, it is possible to use the second irradiation light L_{TOF} emanated from the exit end face 121′b (hereinafter referred to as “second irradiation light L_{TOF1}”) and the second irradiation light L_{TOF} emanated from the exit end face 121″b (hereinafter referred to as “second irradiation light L_{TOF2}”), for distance measurement.

For example, even when distance measurement is difficult with the second irradiation light L_{TOF1} due to projections and depressions of the subject, there is a possibility that it is possible to perform the distance measurement with the second irradiation light L_{TOF2}. The wavelength band of the second irradiation light L_{TOF1} and the wavelength band of the second irradiation light L_{TOF2} may be the same or may be different.

As described above, if the second irradiation light L_{TOF} is emanated from a plurality of exit end faces, it is impossible to measure a precise distance. Thus, emanation of the second irradiation light L_{TOF1} and emanation of the second irradiation light L_{TOF2} are not performed simultaneously.

### (Optical Device 12: Eighteenth Example)

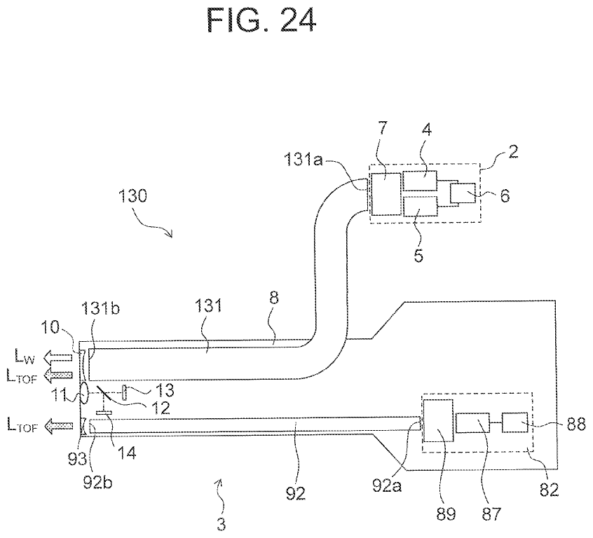

FIG. 24 is a diagram illustrating an optical device of the present embodiment. The same component as that in FIG. 1A and FIG. 18 is denoted by the same numeral and a description thereof is omitted.

An optical device 130 includes the light source unit 2, a second light source unit 82, and the body unit 3. In the optical device 130, the light source unit 2 is disposed at a place away from the body unit 3. The second light source unit 82 is disposed in the interior of the body unit 3.

In the optical device 130, the body unit 3 includes a light guide member 131 and the light guide member 92. The light guide member 131 has an incident end face 131a and an exit end face 131b. The light guide member 92 has the incident end face 92a and the exit end face 92b. The optical device 130 has two incident end faces and two exit end faces.

The light guide member 131 and the light guide member 92 are disposed in the insertion section 8. In the optical device 130, the insertion section 8 has two exit end faces.

The incident end face 131a faces the light collecting unit 7. The incident end face 92a faces the second light collecting unit 89. The exit end face 131b faces the lens 10. The exit end face 92b faces the lens 93.

The first irradiation light L_{W} and the second irradiation light L_{TOF} are incident on the incident end face 131a. Thus, the first irradiation light L_{W} and the second irradiation light L_{TOF} are emanated from the exit end face 131b. Only the second irradiation light L_{TOF} is incident on the incident end face 92a. Thus, only the second irradiation light L_{TOF} is emanated from the exit end face 92b.

In the optical device 130, the second irradiation light L_{TOF} can be emanated from two exit end faces. As a result, in the optical device 130, it is possible to use the second irradiation light L_{TOF} emanated from the exit end face 131b (hereinafter referred to as “second irradiation light L_{TOF3}”) and the second irradiation light L_{TOF} emanated from the exit end face 92b (hereinafter referred to as “second irradiation light L_{TOF4}”), for distance measurement.

For example, even when distance measurement is difficult with the second irradiation light L_{TOF3}, it is possible to perform distance measurement with the second irradiation light L_{TOF4}. The wavelength band of the second irradiation light L_{TOF3} and the wavelength band of the second irradiation light L_{TOF4} may be the same or may be different. Emanation of the second irradiation light L_{TOF3} and emanation of the second irradiation light L_{TOF4} are not performed simultaneously.

In the optical device 120 and the optical device 130, the second irradiation light L_{TOF} can be emanated from the first exit end face and the second exit end face. Thus, it is possible to use at least one of the second irradiation light L_{TOF} emanated from the first exit end face and the second irradiation light L_{TOF} emanated from the second exit end face, for distance measurement.

### (Optical Device 13)

In the optical device of the present embodiment, it is preferable that two or more exit end faces include a first exit end face and a second exit end face, and in the first exit end face and the second exit end face, emanation of the second irradiation light from the first exit end face and emanation of the second irradiation light from the second exit end face be alternately performed.

In the optical device 120 (see FIG. 23A), the first irradiation light L_{W} and the second irradiation light L_{TOF} are emanated from the exit end face 121′b. Only the second irradiation light L_{TOF} is emanated from the exit end face 121″b. The exit end face 121′b is the first exit end face. The exit end face 121″b is the second exit end face. Thus, in the optical device 120, the second irradiation light L_{TOF} can be emanated from both of the first exit end face and the second exit end face.

The distance from the incident end face 121a to the exit end face 121′b is different from the distance from the incident end face 121a to the exit end face 121″b. In this case, there is a time lag between the second irradiation light L_{TOF} emanated from the exit end face 121′b and the second irradiation light L_{TOF} emanated from the exit end face 121″b. Hence, if the subject is irradiated with two rays of the second irradiation light L_{TOF} simultaneously, it is not possible to accurately measure the distance.

In the optical device, it is possible to alternately perform emanation of the second irradiation light L_{TOF} from an exit end face 121′b and emanation of the second irradiation light L_{TOF} from an exit end face 121″b. In this case, the subject is irradiated with one rays of the second irradiation light L_{TOF}. Therefore, it is possible to accurately measure the distance.

Furthermore, there is a possibility that a place that one ray of the second irradiation light L_{TOF} fails to illuminate can be irradiated with the other ray of second irradiation light L_{TOF}. Hence, it is possible to increase the number of measurement sites.

Even in the optical device 130 (see FIG. 24), the second irradiation light L_{TOF} can be emanated from both of the first exit end face and the second exit end face. Thus, the optical device 130 also achieves the same operation effect as the optical device 120 does.

### (Optical Device 14)

In the optical device of the present embodiment, it is preferable that the light intensity be also temporally modulated in the first irradiation light, and the modulation in the first irradiation light and the modulation in the second irradiation light be the same.

In the second irradiation light, light intensity is temporally modulated. This temporal modulation is performed by the light source control unit. In the optical device of the present embodiment, light intensity is temporally modulated also in the first irradiation light, in the same manner as in the second irradiation light.

By doing so, it is possible to suppress occurrence of speckle even when a light source with high coherence is used as the first light source. Thus, it is possible to make uniform illumination in the illumination using the first irradiation light.

### (Optical Device 15)

In the optical device of the present embodiment, it is preferable that return light from the subject be incident on one optical system.

In the optical device 1 (see FIG. 1A), the optical system on which return light from the subject is incident is the optical system 11 alone. In this case, an optical image of the same shape is formed on the first imager 13 and the second imager 14. Hence, no mismatch occurs between image information and distance information. As a result, it is easily associate image information and distance information with each other.

### (Optical Device 16)

In the optical device of the present embodiment, it is preferable that acquisition of image information and acquisition of distance information be performed simultaneously.

It is possible to acquire information in a short time because acquisition of image information and acquisition of distance information can be performed simultaneously.

### (Optical Device 17)

In the optical device of the present embodiment, it is preferable that acquisition of the image information and acquisition of the distance information be alternately performed.

As illustrated in FIG. 14, the second measurement light includes the first irradiation light L_{W} and the second irradiation light L_{TOF} in some cases. In this case, by performing alternate turning-on, acquisition of the image information and acquisition of the distance information are alternately performed.

In this case, it is possible to improve the SN ratio in the second measurement light because the second measurement light includes the second irradiation light L_{TOF} alone. As a result, it is possible to obtain distance information precisely. In addition, it is possible to obtain an image with a good color balance because most of the first irradiation light L_{W} is incident for the first measurement light.

### (Optical Device 18)

In the optical device of the present embodiment, it is preferable that the optical system include a half mirror, and the first measurement light and the second measurement light occur from return light incident on the half mirror.

### (Endoscope System 1)

An endoscope system of the present embodiment includes the optical device described above and a processing device. The processing device includes an assistance information generator configured to generate assistance information. The assistance information is generated based on the image information and the distance information. The assistance information includes information on position and information on shape of a lesion candidate region, and a length between necessary points calculated from the distance information based on the information.

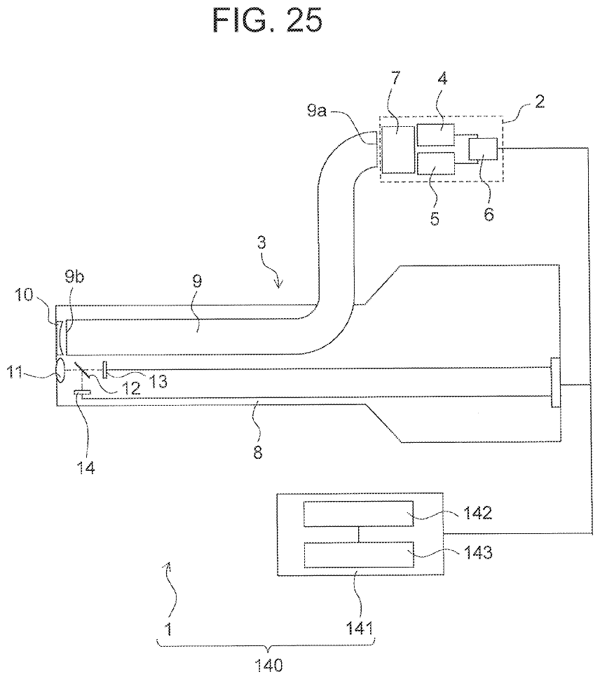

FIG. 25 is a diagram illustrating an endoscope system of the present embodiment. The same component as that in FIG. 1A is denoted by the same numeral and a description thereof is omitted.

An endoscope system 140 includes the optical device 1 and a processing device 141. The processing device 141 includes an image processing circuit 142 and an assistance information generator 143.

In the image processing circuit 142, an assistance image is generated. The assistance image is generated based on image information and distance information.

As described above, the image information is information acquired using the first imager. The first imager includes a plurality of minute light-receiving portions. Each of the light-receiving portions has image information. It is possible to generate an image of the subject from the image information of each light-receiving portion.

The image information is, for example, information on brightness and information on color. Thus, an image obtained from the first imager (hereinafter referred to as “image for observation”) is generated based on information on brightness and information on color.

The distance information is information acquired using the second imager. The second imager includes a plurality of minute light-receiving portions. Each of the light-receiving portions has distance information. It is possible to generate an image of the subject from the distance information of each light-receiving portion.

A region that is presumably a lesion area (hereinafter referred to as “lesion candidate region”) is sometimes included in an image for observation. In this case, the user can mark the lesion candidate region using the assistance image. As just described, it is possible to use the assistance image to display a normal image and specify a lesion candidate region in the normal image.

The assistance image may be arranged separately from the normal image. In this case, a plurality of optional points in the assistance image may be marked. For example, when two points are marked, the distance between the marked two points are displayed on the assistance image. Thus, it is possible to view the image information and the distance on the same image. It is possible to perform marking, for example, by a mouse, eye control, or coordinate input. The marking may be input such that a lesion candidate region is surrounded.

Furthermore, in the assistance image, the lesion candidate region is displayed together with the image for observation. Hence, in the assistance image, it is possible to easily modify the range of marking.

In the assistance information generator 143, assistance information is generated. The assistance information includes at least one of information on position and information on shape of the lesion candidate region, and the length between necessary points calculated from the distance information based on the information. It is possible for the user to use this information as supplementary information for diagnosis of the lesion candidate region.

The endoscope system 140 may include a controller. The controller is used to input the marked region or position, receive the modified information, display the assistance image, display the assistance information, and calculate the distance or size.

### (Endoscope System 2)

In the endoscope system of the present embodiment, it is preferable that an inclination in a pixel of the image for observation be complemented or estimated based on the distance information.

When the pixels of an image for observation and the pixels of a measurement image do not have one-to-one correspondence, the inclination in a pixel of the image for observation may be complemented and estimated using a plurality of pixels of the measurement image.

Furthermore, the distance estimation need not be performed for all the pixels. For example, the distance corresponding to a position specified in advance may be estimated. Alternatively, the distance corresponding to a position representing a specified area may be estimated. It is possible to specify a position or specify an area in advance manually or by artificial intelligence.

### (Endoscope System 3)

An endoscope system of the present embodiment includes the optical device described above and a processing device, an image for observation of a subject is generated based on the image information, a distance or a distance and an inclination in a pixel of the image for observation are complemented and estimated based on the distance information, and length information is acquired from the result of estimation.

In the coaxial optical system illustrated in FIG. 1A, when the number of light-receiving portions of the first imager is equal to the number of light-receiving portions of the second imager, the pixels of the image for observation and the pixels of the measurement image have one-to-one correspondence. In the parallel optical system as illustrated in FIG. 2, for example, magnification or aberration differs between two optical systems. Hence, the pixels of the image for observation and the pixels of the measurement image do not necessarily have one-to-one correspondence.

Furthermore, both in the coaxial optical system and the parallel optical system, the number of light-receiving portions of the second imager is sometimes smaller than the number of light-receiving portions of the first imager. Also in this case, the pixels of the image for observation and the pixels of the measurement image do not have one-to-one correspondence.

When the pixels of the image for observation and the pixels of the measurement image do not have one-to-one correspondence, it is necessary to perform mapping in accordance with a certain rule. In this mapping, a plurality of pixels of the image for observation are associated with one pixel of the measurement image. In this way, the distance in a pixel of the image for observation may be complemented or estimated using a plurality of pixels of the measurement image.

Furthermore, the distance estimation need not be performed for all the pixels. For example, the distance corresponding to a position specified in advance may be estimated. Alternatively, the distance corresponding to a position representing a specified area may be estimated. It is possible to specify a position or specify an area in advance manually or by AI.

### (Endoscope System 4)

In an endoscope system of the present embodiment, it is preferable that identification of a lesion candidate region, identification of a lesion area, modification after the identification, extraction of a lesion area, or diagnosis of a lesion area be performed by artificial intelligence.

It is possible to provide artificial intelligence in the controller. A lesion candidate region can be identified by the user. However, identification of a lesion candidate region may put heavy load on the user.

In this endoscope system, a lesion candidate region is identified by artificial intelligence. Hence, the user only has to determine whether the identified lesion candidate region is appropriate. As a result, it is possible to alleviate the burden on the user. Furthermore, it is possible to identify a lesion candidate region in a short time.

Furthermore, it is also possible to perform identification of a lesion area, modification after the identification, extraction of a lesion area, or diagnosis of a lesion area by artificial intelligence. In any case, the user only has to determine whether the result of the action is appropriate.

In diagnosis by artificial intelligence, it is possible to use a normal image or a special image. The normal image is, for example, an image obtained by illumination with white light. The special image is, for example, an image (NBI image) obtained by illumination with narrow-band light. Image processing may be performed on the normal image or the special image by artificial intelligence.

When a lesion area is identified by artificial intelligence, whether the identification is appropriate is determined by the user. If the identification is not appropriate, a range that identifies a lesion area is modified. If it is determined that the identification is appropriate, the modified lesion area is extracted. For the extracted lesion area, length, for example, the longer diameter of the lesion area or the shorter diameter of the lesion area is calculated based on the distance information. The calculated length is displayed on the assistance image.

The extracted lesion area is diagnosed by artificial intelligence. Thus, it is possible to additionally display the diagnosis result together with the length on the assistance image.

If the processing time by artificial intelligence is sufficiently short, it is possible to perform calculation of the length and diagnosis of the lesion area before modification. Then, the calculation result of the length and the diagnosis result of the lesion area may be updated in accordance with the result of modification, and the updated result may be displayed.

The present disclosure provides an optical device and an endoscope system with which error information included in distance information is reduced.

As described above, the present disclosure is suitable for an optical device and an endoscope system with which error information included in distance information is reduced.

What is claimed is:

1. An optical device comprising:
a light source unit; and a body unit, wherein
the light source unit includes
a first light source configured to emit first irradiation light,
a second light source configured to emit second irradiation light,
a light source control unit configured to control the first light source and the second light source, and
a light collecting unit on which the first irradiation light and the second irradiation light are incident,
the body unit includes a rigid and tubular insertion section or a flexible and tubular insertion section,
the insertion section includes
a light guide member formed of a transparent medium having a refractive index larger than 1,
an optical system on which return light from a subject is incident,
a first imager configured to output image information of the subject based on first measurement light, and
a second imager configured to output distance information from the optical system to the subject based on second measurement light,
in the second irradiation light, light intensity is temporally modulated,
the light guide member has an incident end face positioned on a light collecting unit side and an exit end face positioned on a subject side,
third irradiation light emanated from the light collecting unit is emanated from the insertion section toward the subject,
the first measurement light includes light in a same wavelength band as a part of a wavelength band of the first irradiation light,
the second measurement light includes light in a same wavelength band as a wavelength band of the second irradiation light, and
an incident angle of the second irradiation light at an incident end face on which the second irradiation light is incident is smaller than an incident angle of the first irradiation light at an incident end face on which the first irradiation light is incident.

2. The optical device according to claim 1, wherein the second irradiation light is light in a wavelength band on a shorter wavelength side than an infrared wavelength band.

3. The optical device according to claim 2, wherein the second irradiation light includes a wavelength band of 460 nm or more and 510 nm or less.

4. The optical device according to claim 2, wherein the second irradiation light is 460 nm or more and 510 nm or less.

5. The optical device according to claim 2, wherein a wavelength band of the second irradiation light includes a wavelength band in which absorption in hemoglobin is large.

6. The optical device according to claim 2, wherein the second irradiation light is ultraviolet light.

7. The optical device according to claim 1, wherein the incident angle of the second irradiation light is 5.7° or less.

8. The optical device according to claim 1, wherein turning-on of the first light source and turning-on of the second light source are alternately performed.

9. The optical device according to claim 1, wherein
the insertion section has one incident end face,
the one incident end face includes a first incident region and a second incident region, and
the first irradiation light is incident on the first incident region, and the second irradiation light is incident on the second incident region.

10. The optical device according to claim 1, wherein
the insertion section has a plurality of incident end faces,
the incident end faces are spatially separated, and
an incident end face on which the first irradiation light is incident is different from an incident end face on which the second irradiation light is incident.

11. The optical device according to claim 10, wherein the second light source is disposed in the body unit.

12. The optical device according to claim 11, wherein an incident angle of the second irradiation light at an incident end face on which the second irradiation light is incident is 9.9° or less.

13. The optical device according to claim 11, wherein an area of the second incident end face is smaller than an area of the first incident end face.

14. The optical device according to claim 1, wherein
the insertion section has a plurality of exit end faces, and
the second irradiation light is emanated only from predetermined substantially one exit end face.

15. The optical device according to claim 1, wherein
the insertion section has a plurality of exit end faces, and
the second irradiation light is emanated from two or more exit end faces, and the second irradiation light is emanated only from one exit end face at a same time.

16. The optical device according to claim 15, wherein
the two or more exit end faces include a first exit end face and a second exit end face, and
at the first exit end face and the second exit end face, emanation of the second irradiation light from the first exit end face and emanation of the second irradiation light from the second exit end face are alternately performed.

17. The optical device according to claim 1, wherein
in the first irradiation light, light intensity is also temporally modulated, and
modulation in the first irradiation light and modulation in the second irradiation light are same.

18. The optical device according to claim 1, wherein acquisition of the image information and acquisition of the distance information are alternately performed.

19. An endoscope system comprising:
the optical device according to claim 1; and a processing device, wherein
the processing device includes an assistance information generator configured to generate assistance information,
the assistance information is generated based on the image information and the distance information, and
the assistance information includes information on position and information on shape of a lesion candidate region, and a length between necessary points calculated from the distance information based on the information.

20. An endoscope system comprising:
the optical device according to claim 1; and a processing device, wherein
an image for observation of the subject is generated based on the image information,
a distance or a distance and an inclination in a pixel of the image for observation are complemented and estimated based on the distance information, and
length information is acquired from a result of the estimation.

21. The endoscope system according to claim 19, wherein identification of a lesion candidate region, identification of a lesion area, modification after the identification, extraction of a lesion area, or diagnosis of a lesion area is performed by artificial intelligence.

22. The endoscope system according to claim 20, wherein identification of a lesion candidate region, identification of a lesion area, modification after the identification, extraction of a lesion area, or diagnosis of a lesion area is performed by artificial intelligence.

23. An optical device comprising:
a first light source configured to emit first irradiation light for acquiring image information of a subject;
a second light source configured to emit second irradiation light for acquiring distance information from an optical system to the subject;
a light collecting unit on which the first irradiation light and the second irradiation light are incident, the light collecting unit being configured to collect light on an incident end face of a light guide member; and
a light source control unit configured to control the first light source and the second light source,
wherein an incident angle of the second irradiation light at an incident end face on which the second irradiation light is incident is smaller than an incident angle of the first irradiation light at an incident end face on which the first irradiation light is incident.

24. The optical device according to claim 23, wherein an incident angle of the second irradiation light at an incident end face on which the second irradiation light is incident is 5.7° or less.

25. The optical device according to claim 23, wherein an incident angle of the second irradiation light at an incident end face on which the second irradiation light is incident is 9.9° or less.

26. The optical device according to claim 25, wherein an area of the second incident end face is smaller than an area of the first incident end face.

27. The optical device according to claim 23, wherein a wavelength band of the second irradiation light includes a wavelength band in which absorption in hemoglobin is large.

28. The optical device according to claim 23, wherein acquisition of the image information and acquisition of the distance information are alternately performed.

29. An endoscope system comprising:
the optical device according to claim 23; and a processing device, wherein
the processing device includes an assistance information generator configured to generate assistance information,
the assistance information is generated based on the image information and the distance information, and
the assistance information includes information on position and information on shape of a lesion candidate region, and a length between necessary points calculated from the distance information based on the information.

30. An endoscope system comprising:
the optical device according to claim 23; and a processing device, wherein
an image for observation of the subject is generated based on the image information,
a distance or a distance and an inclination in a pixel of the image for observation are complemented and estimated based on the distance information, and
length information is acquired from a result of the estimation.

31. A method of collecting light to an incident end face of a light guide member, the method comprising:
emitting, by a first light source, first irradiation light for acquiring image information of a subject;
emitting, by a second light source, second irradiation light for acquiring distance information from an optical system to the subject; and
collecting, by a light collecting unit, the first irradiation light and the second irradiation light incident on the light collecting unit, to an incident end face of a light guide member,
wherein an incident angle of the second irradiation light at an incident end face on which the second irradiation light is incident is smaller than an incident angle of the first irradiation light at an incident end face on which the first irradiation light is incident.
